(19) United States
(12) Patent Application Publication
(10) Pub. No.: US 2007/0048848 A1
(43) Pub. Date: Mar. 1, 2007

(54) Method, apparatus and system for biodiesel production from algae

(75) Inventor: James T. Sears, Boulder, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: SUNSOURCE INDUSTRIES

(21) Appl. No.: 11/510,148

(22) Filed: Aug. 24, 2006

Related U.S. Application Data
(60) Provisional application No. 60/711,316, filed on Aug. 25, 2005.
(60) Provisional application No. 60/733,569, filed on Nov. 4, 2005.
(60) Provisional application No. 60/740,855, filed on Nov. 30, 2005.
(60) Provisional application No. 60/757,587, filed on Jan. 10, 2006.
(60) Provisional application No. 60/818,102, filed on Jun. 30, 2006.

(51) Int. Cl. C12P 7/64; C12N 1/12; C07C 51/43

(52) U.S. Cl. 435/134; 435/257.1; 554/174

(57) ABSTRACT

The present disclosure concerns methods, apparatus, compositions and systems relating to closed bioreactors for algal culture and harvesting. In certain embodiments, the system may comprise bags with various layers, including a thermal barrier layer, that may be used to contain the algal culture and/or to thermally regulate the temperature of the algal culture. The system may comprise various mechanisms for moving fluid within the system, such as a roller type mechanism, and may provide temperature regulation by compartmentalization of the fluid to regulate absorption of solar radiation and/or conductive or emissive heat loss and gain. Various mechanisms may be used to harvest and process the algae and/or to convert algal oil into biodiesel and other products.

### RELATED APPLICATIONS

The present application claims priority under 35 U.S.C. 119(e) to Provisional U.S. Patent Application Ser. Nos. 60/711,316, filed Aug. 25, 2005; 60/733,569, filed Nov. 4, 2005; 60/740,855, filed Nov. 30, 2005; 60/757,587, filed Jan. 10, 2006; and 60/818,102, filed Jun. 30, 2006; each incorporated herein by reference in its entirety.

### FIELD

The present invention relates to methods, compositions, apparatus and a system for growing and harvesting algae and/or other aquatic organisms. Certain embodiments concern methods, compositions, apparatus and a system for production of useful products from algae, such as biofuels (e.g., biodiesel, methanol, ethanol), bio-polymers, chemical precursors and/or animal or human food. Other embodiments concern use of such a system to remove carbon dioxide from sources such as power plant emissions.

### BACKGROUND

In 1996 the National Renewable Energy Laboratory (NREL) in Golden, Colorado was forced to abandon its 10 year $25 million Aquatic Species Program that focused on extracting biodiesel from unusually productive species of algae. Before losing funding, the government scientists had demonstrated oil production rates 200 times greater per acre than achievable with fuel production from soybean farming. However, three fundamental problems limited the commercialization potential of algal culture.

The three problems were: [1] Oil prices were low in 1996 and hard to compete against. [2] The oil rich algae were difficult to protect from consumption or displacement by invading organisms as they were grown in ponds open to the environment. [3] Algae best produce oil within a narrow temperature band, yet night sky radiation and low temperatures and high temperature days and excessive solar IR radiation interfered with NREL's pond experiments by wildly varying the cultivation temperature.

A need exists in the field for technologies and methods to address these issues and provide a competitively priced, algal culture based biodiesel production in a biologically closed system, with better temperature control than the open pond model.

### SUMMARY

In certain embodiments, the methods, compositions, apparatus and system disclosed and claimed herein provide for biodiesel production from algal culture that is priced at or below diesel fuel costs from petroleum based production. The closed culture and harvesting system greatly reduces problems from contaminating algae, algae consuming microorganisms and/or other extraneous species. In more preferred embodiments, the apparatus is designed to be installed and operated in an outdoor environment, where it is exposed to environmental light, temperature and weather. The apparatus, system and methods provide for improved thermal regulation designed to maintain temperature within the range compatible with optimal growth and oil production. Another advantage of the system is that it may be constructed and operated on land that is marginal or useless for cultivation of standard agricultural crops, such as corn, wheat, soybeans, canola or rice.

The disclosed bioreactor technology stabilizes algae cultivation temperature with low energy usage, practical on any scale. By solving the problems of temperature and invading species at an affordable cost and adding a few other technologies, we have developed a system that is useful for creating a host of high value products from algae that is largely fed by industrial, agricultural, and municipal waste products. In some embodiments, the algal culture may be used directly to provide an animal or human food source, for example by culturing edible algae such as Spirulina. In other embodiments, the algal culture may be used to support growth of a secondary food source, such as shrimp or other aquatic species that feed on algae. Methods of shrimp farming and aquaculture of other edible species are known in the art and may utilize well-characterized species such as Penaeus japonicus, Penaeus duorarum, Penaeus aztecus, Penaeus setiferus, Penaeus occidentalis, Penaeus vannamei or other peneid species. The skilled artisan will realize that this disclosure is not limiting and other edible species that feed on algae may be grown and harvested.

One embodiment concerns methods, an apparatus and a system for producing biodiesel. High oil strains of algae are cultured in a closed system and harvested. Algae are completely or partially separated from the medium, which may be filtered, sterilized and reused. The oil is separated from the algal cells and processed into diesel using standard transesterification technologies such as the well-known Connemann process (see, e.g., U.S. Pat. No. 5,354,878, the entire text of which is incorporated herein by reference). However, it is contemplated that any known methods for converting algal oil products into biodiesel may be used.

In other embodiments, the system, apparatus and methods are of use for removing carbon dioxide pollution, for example from the exhaust gases generated by power plants, factories and/or other fixed source generators of carbon dioxide. The CO₂ may be introduced into the closed system bioreactor, for example by bubbling through the aqueous medium. In a preferred embodiment, CO₂ may be introduced by bubbling the gas through a perforated neoprene membrane, which produces small bubbles with a high surface to volume ratio for maximum exchange. In a more preferred embodiment, the gas bubbles may be introduced at the bottom of a water column in which the water flows in the opposite direction to bubble movement. This counterflow arrangement also maximizes gas exchange by increasing the time the bubbles are exposed to the aqueous medium. To further increase CO₂ dissolution, the height of the water column may be increased to lengthen the time that bubbles are exposed to the medium. The CO₂ dissolves in water to generate H₂CO₃, which may then be “fixed” by photosynthetic algae to produce organic compounds. It is estimated that the system and apparatus disclosed herein, installed over a surface area of about 60 square miles (4.5 mile radius), would fix sufficient CO₂ to completely scrub the carbon exhaust of a 1 gigawatt power plant. At the same time, the carbon dioxide would provide an essential nutrient to support algal growth. Such an installation would produce algal lipid plus carbohydrate co-products that could generate about 14,000 gal/acre/year of total fuel output, absorbing 6 million tons/year of generated CO₂ from the power plant. The value of the generated biodiesel plus methane produced by anarobically digesting the carbohydrate fraction of the algae plus potential carbon credits generated would produce a net profit of more than twice the value of the electrical energy generated by a typical coal or natural gas fired power plant.

Although there are thousands of species of known naturally occurring algae, any one of which may be used for biodiesel production and formation of other products, in certain embodiments the algae may be genetically engineered to further increase biodiesel feedstock production per unit acre. The genetic modification of algae for specific product outputs is relatively straight forward using techniques well known in the art. However, the low-cost methods for cultivation, harvesting, and product extraction disclosed herein may be used with either transgenic or non-transgenic algae. The skilled artisan will realize that different algal strains will exhibit different growth and oil productivity and that under different conditions, the system may contain a single strain of algae or a mixture of strains with different properties, or strains of algae plus symbotic bacteria. The algal species used may be optimized for geographic location, temperature sensitivity, light intensity, pH sensitivity, salinity, water quality, nutrient availability, seasonal differences in temperature or light, the desired end products to be obtained from the algae and a variety of other factors.

The disclosed closed bioreactor system and methods are scalable to any level of production desired, resulting in biodiesel feedstock production at well under current wholesale prices; even without factoring in government subsidies for biodiesel fuels.

Some embodiments may concern apparatus, methods and systems for temperature control of the algal culture. In one preferred embodiment, the closed bioreactor is comprised of flexible plastic tubes with an adjustable thermal barrier layer. The tubes and thermal barrier may be constructed of a variety of materials, such as polyethylene, polypropylene, polyurethane, polycarbonate, polyvinylpyrrolidone, polyvinylchloride, polystyrene, poly(ethylene terephthalate), poly(ethylene naphthalate), poly(1,4-cyclohexane dimethylene terephthalate), polyolefin, polybutylene, polyacrylate and polyvinlyidene chloride. In embodiments involving culture of photosynthetic algae or organisms that are fed on algae, the material of the thermal barrier preferably exhibits a transmission of visible light in the red and blue wavelengths of at least 50%, preferably over 60%, more preferably over 75%, more preferably over 90%, more preferably over 90%, most preferably about 100%. In other preferred embodiments, the material used for the top surface of the tubes exhibits a transmission of visible light of at least 90%, more preferably over 95%, more preferably over 98%, most preferably about 100%. In preferred embodiments polyethylene is used. Polyethylene transmits both long-wave black body radiation and red and blue visible light, allowing the temperature control system to radiate the inner heat of the water to the night sky and allowing the algae to receive visible light to support photosynthesis whether the medium is above or below the thermal barrier. Polyethylene exhibits increased transmittance of long wave infrared light associated with room temperature blackbody radiation, in comparison to certain alternative types of plastic. In various embodiments, thin layers of UV blocking materials may be applied to the surface of the tubes to reduce UV-degradation of the plastic. In other embodiments, fluorescent dyes that convert infrared (IR) or ultraviolet (UV) light to the visible (photosynthetic) light spectrum may be incorporated into the tube to increase efficiency of solar energy capture by photosynthetic organisms. Such dyes are known in the art, for example for coating the glass or plastic surfaces of greenhouses, or in fluorescent lighting systems that convert UV to visible light wavelengths. (See, e.g., Hemming et al., 2006, Eur. J. Hort. Sci. 71(3); Hemming et al., in International Conference on Sustainable Greenhouse Systems, (Straten et al., eds.) 2005.)

In embodiments employing a thermal barrier within the tubes, the aqueous medium containing the algae may be directed either above or below the thermal barrier. Under conditions of low temperature, the liquid may be directed above the thermal barrier, where it is exposed to increased solar irradiation including the infared wavelengths, resulting in temperature increase. Under high temperature conditions, the liquid may be directed below the thermal barrier, where it is partially shielded from solar irradiation and simultaneously may lose heat by contact with the underlying ground layer. In still other embodiments, the ground underlaying the closed bioreactor may be used as a heat sink and/or heat source, storing heat during the day and releasing it at night.

When the thermal barrier is up (at the top of the tube), the liquid in the tubes is isolated from both radiative and conductive heat transfer to the outside environment. However, it is in intimate thermal contact with the ground underneath. When the thermal barrier is down the liquid may easily gain or lose heat to the environment via both radiation and conduction. In effect, the thermal barrier acts as a thermal switch that can be used to take advantage of opportune environmental conditions like night, day, rain, clouds, etc. to gain or shed heat to control the temperature of the fluid. The ground beneath the apparatus has thermal mass whose temperature can also be modulated by close thermal contact when the thermal barrier is in the up position. The heat energy in this thermal mass may be used to further control the temperature of the fluid. If a cold night is anticipated, the fluid can be allowed to warm to slightly above optimum temperature during the day with the thermal barrier in the down position. Shift of the thermal barrier to the up position transfers this positive heat energy to the ground thermal mass. Several cycles of fluid warming and ground heating may occur. The heat transferred into the ground thermal mass may then be transferred back to the liquid during a cold night by keeping the thermal barrier is in the up position, to stabilize the water temperature in an optimal range.

Alternatively, when an excessively hot day is anticipated, the barrier may be placed in the down position at night until the mixture is slightly below the optimum temperature and then shifted to the upper position, where the cooled water is in contact with the ground, to pump down the temperature of the ground. This cycle may be repeated several times during the night. As the ensuing day heats up, the thermal barrier is raised, thereby connecting the fluid thermally to the ground to lengthen the time that the fluid stays at an acceptably low temperature.

Other embodiments may comprise apparatus and methods for liquid circulation within and extraction of oxygen or other gases from the closed bioreactor. In a preferred embodiment, large rollers may be arranged to roll over the surface of the closed tubes, pushing liquid along the bag. In addition to moving fluid, the rollers would function to collect bubbles of dissolved gases, such as oxygen that is generated by photosynthetic organisms, which may be removed from the system to reduce oxygen inhibition of algal growth. Because the roller compression does not extend all the way to the bottom of the tube, the roller movement creates a high-velocity localized “backwash” immediately under the roller that serves to scrub the lower tube surface to reduce attachment to and biofouling of the tube surface and to resuspend organisms that have settled to the bottom of the tube. Similarly, the movement of the accumulated gas bubble and gas/water interface in front of the roller at the top of the tube also scrubs the upper tube surface, reducing biofilm formation and increasing light transmission through the top surface. The roller system is a preferred method to move fluid through the tubes while minimizing hydrodynamic shear that would inhibit aquatic organism growth and division. Another benefit of the roller system is that when fluid is being diverted from below to above the thermal barrier, the roller provides a low-energy mechanism for moving a buoyant thermal barrier to the bottom of the tube, as the roller semi-seals the barrier to the tube bottom as it rolls along the tube.

Collection systems, such as sippers, may be arranged to siphon concentrated suspensions of oil-containing algae out of the system. In a more preferred embodiment, the hydrodynamic flow through the bioreactor is designed to produce a “whirlpool” effect, for example in a chamber at one end of the bags. The whirlpool results in a concentration of algae and partial separation from the liquid medium, allowing more efficient harvesting, or to remove undesired byproducts of metabolism like dead cells and mucilage containing bacteria. Other mechanisms for adding nutrients and/or removing waste products from the closed bioreactor may also be provided. One or more sipper tubes may be operably coupled to the whirlpool system to increase efficiency of harvesting from and/or nutrient input to the apparatus.

Certain embodiments may concern axial vortex inducers to provide for rotation of the algae suspension volume to within the top inch of the bioreactor which in a dense aquaculture may be the only volume that receives significant levels of photosynthetic light. The rotation of the water column within the tube results in the periodic movement of organisms between the light-rich environment at the top of the tube and dark regions at the bottom of the tube. In a preferred embodiment, the flexible tubes containing the algae are about 12 inches in height. At high algal density, sunlight will only penetrate approximately the top 1 inch layer of the suspension. Without a mechanism for rotation of the water column, aquatic organisms in the top inch would be overexposed to sunlight and aquatic organisms in the bottom 11 inches would be underexposed. In a preferred embodiment, the axial vortex inducers comprise internal flow deflectors (structured axial flow rotators) within the flexible plastic tubes, discussed below.

In an exemplary embodiment, the deflectors may comprise 6 inch wide by 12 inches long strips of flexible plastic tapered to 2 inches in the middle extending vertically through the tube, with a ninety degree twist from the top to bottom of the strip. In the exemplary illustration of FIG. 17B, the strips are viewed edge on so that the 2 inch middle width is not apparent. The strips may be arranged, for example, at intervals of about 1 foot spacing across the width of the tube (square propellers, defined as a propeller whose pitch=its diameter. In this exemplary illustration, when fluid flows through the tube construction the contained algae in a tube 1 foot thick would move forward in a helical spiral with a rotational period of 3.14 feet longitudinally. Considering a row of strips extending across the width of the tube, alternating strips would exhibit a clockwise or counterclockwise rotation. From the perspective of a column of water moving down the long axis of the tube, a single column would rotate either clockwise or counterclockwise down the entire length of the tube, while adjacent columns would exhibit the opposite rotation. This would minimize frictional induced turbulence between adjacent columns of water. The width, degree of rotation and spacing of the strips, including the spacing between adjacent rows of strips, may be adjusted to optimize structured low-friction, low-random turbulence axial rotation of individual algae cells in and out of the high light zone. In embodiments utilizing an internal thermal barrier within the tubes, one set of axial vortex inducers may be arranged on one side of the thermal barrier and another set on the other side of the barrier. Since turbulence would be minimized by extension of the axial vortex inducers, it is anticipated that where an internal thermal barrier is used the diversion of fluid would be directed so that the majority of water flow, preferably about 90% or more, is directed either above or below the thermal barrier. In this configuration, one set of axial vortex inducers would be folded in between the thermal barrier and the top or bottom of the tube, while the other set would be fully extended. While these axial vortex inducers are envisioned as flexible strips of 0.01″ thick polyethelene, they could also be stiffer hinged plastic constructions or even directional tabs or hoops that protrude from the inner surface of the bags and thermal barrier layer without actually connecting one layer to the other. In all cases the directional elements are arranged to create counter rotating axial flows with a side by side periodicity approximately equal to the height of the bag channel. A model for water flow induced by the axial vortex inducers is exemplified in FIG. 17A-B.

In some embodiments, the emissivity properties of the thermal barrier may be adjusted by incorporation of other materials of selected optical characteristics. For example, quartz sand from specific sources may have desirable optical properties and could be embedded within the upper surface of the thermal barrier. (See, e.g., FIG. 10.) Alternatively, doped glass or quartz beads or ceramic tiles of selected optical properties might be embedded within the upper surface of the thermal barrier. FIG. 11 shows an exemplary optical transmittance profile for an idealized thermal barrier. Current thermal barrier material in use (foamed polyethylene) passes about 60% of photosynthetic light and materials transmitting 75% or more may be utilized.

Various embodiments may concern apparatus and methods for modeling algal production under environmental conditions. An example of a remote sensing bioreactor for condition optimization and algal strain selection is shown in FIG. 8.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 Exemplary system schematic

FIG. 2 Exemplary aquaculture farm view from sky

FIG. 3 Exemplary bioreactor with rollers and harvesting vortexes

FIG. 4 Exemplary thermal control system

FIG. 5 Exemplary bio-fouling countermeasure (nano coating)

FIG. 6 Continuous flow autoclave

FIG. 7 Exemplary extraction roller

FIG. 8 Exemplary remote driven bioreactor technology

FIG. 9 Alternative two-bag system for bioreactor

FIG. 10 Emissivity profile of sand sample obtained from Goleta Beach, Calif.

FIG. 11 Exemplary transmittal profile of idealized material for thermal barrier

FIG. 12 Exemplary CO₂ bubbler for gas dissolution

FIG. 13 Model for exemplary whirlpool device

FIG. 14 Further detail of exemplary whirlpool device, showing dwell tube and speed up cone and stator fins

FIG. 15A Fluid mechanics of whirlpool device

FIG. 15B Whirlpool with sipper tubes

FIG. 16 Computer simulation of water temperature in closed bioreactor with and without barrier

FIG. 17 Water flow induced by exemplary axial vortex inducers

FIG. 18 Model ⅕ scale closed system exemplary bioreactor

FIG. 19 Exemplary roller, side walls and end chamber with CO₂ bubbler

FIG. 20 Exemplary roller, side walls and end chamber to contain whirlpool device

FIG. 21 Preferred embodiment of the flow bypass for bidirectional roller system

FIG. 22 Exemplary “belly pan” for bidirectional roller system

FIG. 23 Illustrative embodiment of whirlpool device

FIG. 24 Example of flexible tube construction and attachment mechanism

FIG. 25 Example of preferred roller drive system

FIG. 26 Exemplary reactor bag sidewall design

FIG. 27 Exemplary bioreactor apparatus controller system

FIG. 28 Exemplary control cycle

FIG. 29 Exemplary Frenel pattern for tube top surface

### DESCRIPTION OF ILLUSTRATIVE EMBODIMENTS

Terms that are not otherwise defined herein are used in accordance with their plain and ordinary meaning.

As used herein, “a” or “an” may mean one or more than one of an item.

As used herein, “about” means plus or minus ten percent. E.g., “about 100” refers to any number between 90 and 110.

Transgenic Algae for Enhanced Oil Production

In certain embodiments, algae of use to produce biodiesel may be genetically engineered (transgenic) to contain one or more isolated nucleic acid sequences that enhance oil production or provide other characteristics of use for algal culture, growth, harvesting or use. Methods of stably transforming algal species and compositions comprising isolated nucleic acids of use are well known in the art and any such methods and compositions may be used in the practice of the present invention. Exemplary transformation methods of use may include microprojectile bombardment, electroporation, protoplast fusion, PEG-mediated transformation, DNA-coated silicon carbide whiskers or use of viral mediated transformation (see, e.g., Sanford et al., 1993, Meth. Enzymol. 217:483-509; Dunahay et al., 1997, Meth. Molec. Biol. 62:503-9; U.S. Pat. Nos. 5,270,175; 5,661,017, incorporated herein by reference).

For example, U.S. Pat. No. 5,661,017 discloses methods for algal transformation of chlorophyll C-containing algae, such as the Bacillariophyceae, Chrysophyceae, Phaeophyceae, Xanthophyceae, Raphidophyceae, Prymnesiophyceae, Cryptophyceae, Cyclotella, Navicula, Cylindrotheca, Phaeodactylum, Amphora, Chaetoceros, Nitzschia or Thalassiosira. Compositions comprising nucleic acids of use, such as acetyl-CoA carboxylase, are also disclosed.

In various embodiments, a selectable marker may be incorporated into an isolated nucleic acid or vector to select for transformed algae. Selectable markers of use may include neomycin phosphotransferase, aminoglycoside phosphotransferase, aminoglycoside acetyltransferase, chloramphenicol acetyl transferase, hygromycin B phosphotransferase, bleomycin binding protein, phosphinothricin acetyltransferase, bromoxynil nitrilase, glyphosate-resistant 5-enolpyruvylshikimate-3-phosphate synthase, cryptopleurine-resistant ribosomal protein S14, emetine-resistant ribosomal protein S14, sulfonylurea-resistant acetolactate synthase, imidazolinone-resistant acetolactate synthase, streptomycin-resistant 16S ribosomal RNA, spectinomycin-resistant 16S ribosomal RNA, erythromycin-resistant 23S ribosomal RNA or methyl benzimidazole-resistant tubulin. Regulatory nucleic acid sequences to enhance expression of a transgene are known, such as C. cryptica acetyl-CoA carboxylase 5′-untranslated regulatory control sequence, a C. cryptica acetyl-CoA carboxylase 3′-untranslated regulatory control sequence, and combinations thereof.

Separation of Algae and Extraction of Oil

In various embodiments, algae may be separated from the medium and various algal components, such as oil, may be extracted using any method known in the art. For example, algae may be partially separated from the medium using a standing whirlpool circulation, harvesting vortex and/or sipper tubes, as discussed below. Alternatively, industrial scale commercial centrifuges of large volume capacity may be used to supplement or in place of other separation methods. Such centrifuges may be obtained from known commercial sources (e.g., Cimbria Sket or IBG Monforts, Germany; Alfa Laval A/S, Denmark). Centrifugation, sedimentation and/or filtering may also be of use to purify oil from other algal components. Separation of algae from the aqueous medium may be facilitated by addition of flocculants, such as clay (e.g., particle size less than 2 microns), aluminum sulfate or polyacrylamide. In the presence of flocculants, algae may be separated by simple gravitational settling, or may be more easily separated by centrifugation. Flocculent-based separation of algae is disclosed, for example, in U.S. Patent Appl. Publ. No. 20020079270, incorporated herein by reference.

The skilled artisan will realize that any method known in the art for separating cells, such as algae, from liquid medium may be utilized. For example, U.S. Patent Appl. Publ. No. 20040121447 and U.S. Pat. No. 6,524,486, each incorporated herein by reference, disclose a tangential flow filter device and apparatus for partially separating algae from an aqueous medium. Other methods for algal separation from medium have been disclosed in U.S. Pat. Nos. 5,910,254 and 6,524,486, each incorporated herein by reference. Other published methods for algal separation and/or extraction may also be used. (See, e.g., Rose et al., Water Science and Technology 1992, 25:319-327; Smith et al., Northwest Science, 1968, 42:165-171; Moulton et al., Hydrobiologia 1990, 204/205:401-408; Borowitzka et al., Bulletin of Marine Science, 1990, 47:244-252; Honeycutt, Biotechnology and Bioengineering Symp. 1983, 13:567-575).

In various embodiments, algae may be disrupted to facilitate separation of oil and other components. Any method known for cell disruption may be utilized, such as ultrasonication, French press, osmotic shock, mechanical shear force, cold press, thermal shock, rotor-stator disruptors, valve-type processors, fixed geometry processors, nitrogen decompression or any other known method. High capacity commercial cell disruptors may be purchased from known sources. (E.g., GEA Niro Inc., Columbia, Md.; Constant Systems Ltd., Daventry, England; Microfluidics, Newton, Mass.) Methods for rupturing microalgae in aqueous suspension are disclosed, for example, in U.S. Pat. No. 6,000,551, incorporated herein by reference.

Conversion of Algae into Biodiesel

A variety of methods for conversion of photosynthetic derived materials into biodiesel are known in the art and any such known method may be used in the practice of the instant invention. For example, the algae may be harvested, separated from the liquid medium, lysed and the oil content separated. The algal-produced oil will be rich in triglycerides. Such oils may be converted into biodiesel using well-known methods, such as the Connemann process (see, e.g., U.S. Pat. No. 5,354,878, incorporated herein by reference). Standard transesterification processes involve an alkaline catalyzed transesterification reaction between the triglyceride and an alcohol, typically methanol. The fatty acids of the triglyceride are transferred to methanol, producing alkyl esters (biodiesel) and releasing glycerol. The glycerol is removed and may be used for other purposes.

Preferred embodiments may involve the use of the Connemann process (U.S. Pat. No. 5,354,878). In contrast to batch reaction methods (e.g., J. Am. Oil Soc. 61:343, 1984), the Connemann process utilizes continuous flow of the reaction mixture through reactor columns, in which the flow rate is lower than the sinking rate of glycerine. This results in the continuous separation of glycerine from the biodiesel. The reaction mixture may be processed through further reactor columns to complete the transesterification process. Residual methanol, glycerine, free fatty acids and catalyst may be removed by aqueous extraction. The Connemann process is well-established for production of biodiesel from plant sources such as rapeseed oil and as of 2003 was used in Germany for production of about 1 million tons of biodiesel per year (Bockey, “Biodiesel production and marketing in Germany,”

www.projectbiobus.com/IOPD_E_RZ.pdf).

However, the skilled artisan will realize that any method known in the art for producing biodiesel from triglyceride containing oils may be utilized, for example as disclosed in U.S. Pat. Nos. 4,695,411; 5,338,471; 5,730,029; 6,538,146; 6,960,672, each incorporated herein by reference. Alternative methods that do not involve transesterification may also be used. For example, by pyrolysis, gasification, or thermochemical liquefaction (see, e.g., Dote, 1994, Fuel 73:12; Ginzburg, 1993, Renewable Energy 3:249-52; Benemann and Oswald, 1996, DOE/PC/93204-T5).

Other Algal Products

In certain embodiments, the disclosed methods, compositions and apparatus may be used for culture of animal or human-edible algae. For example, Spirulina is a planktonic blue-green algae that is rich in nutrients, such as protein, amino acids, vitamin B-12 and carotenoids. Human consumption of Spirulina grown in algae farms amounts to more than one thousand metric tons annually. The skilled artisan will realize that any type of free-living algae may be grown, harvested and utilized by the claimed system, including edible algae like Spirulina, Dunaliella or Tetraselmis (see U.S. Pat. Nos. 6,156,561 and 6,986,323, each incorporated herein by reference.)

Other algal-based products may also be produced using the claimed methods, apparatus and system. For example, U.S. Pat. No. 5,250,427, incorporated herein by reference, discloses methods for photoconversion of organic materials such as algae into biologically-degradable plastics. Any such known method for producing useful products by culture of either normal or transgenic algae may be used.

### EXAMPLES

The methods, compositions, apparatus and system disclosed and claimed herein concern technology that supports large scale and low cost cultivation and harvesting of water born algal cultures. This technology may be used to support industrial manufacturing of the various products that different species of algae can provide. This technology may be of use to economically support the massive cultivation and harvesting of algae. The disclosed apparatus is generally referred to herein as a “bioreactor,” “photo-bioreactor,” “closed system bioreactor” and/or “bioreactor apparatus”. Other machinery, apparatus and/or technologies of use with the bioreactor may include sterilization technology, CO₂ infusion technology, and/or extraction technology.

### Example 1

### Bioreactor System

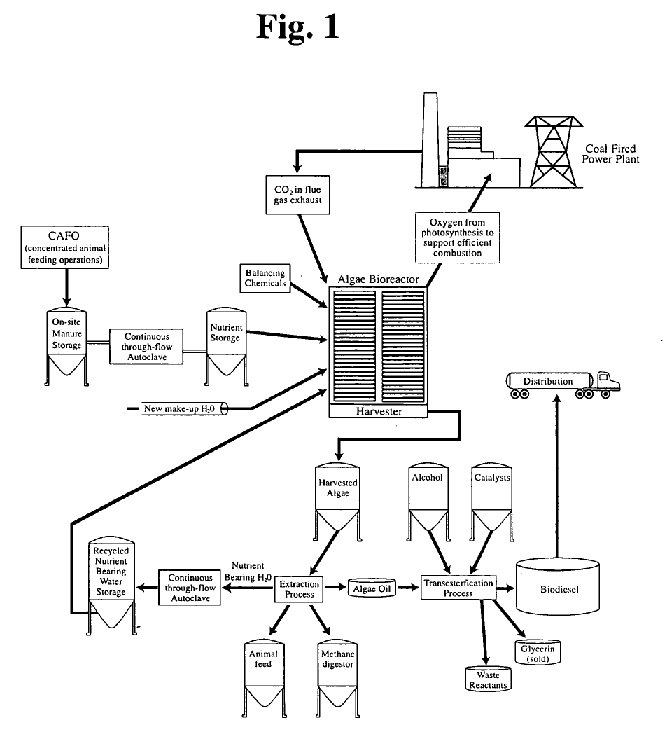

FIG. 1 illustrates an exemplary System Schematic. Elements of the exemplary system include Bioreactor technology, Harvesting technology, Sterilization technology, CO₂ infusion technology, Extraction technology, Remote driven bioreactor technology. As illustrated in FIG. 1, the algal culture operation may derive nutrients from animal feeding operations, such as pig manure. After processing and sterilization, such organic nutrients may be stored and/or added to the culture medium to support algal growth. Since photosynthetic algae “fix” CO₂ for conversion into organic carbon compounds, a CO₂ source, for example the gas exhaust from a power plant, may be utilized to add dissolved CO₂ to the culture medium. CO₂ and nutrients may be utilized by algae to produce oil and other biological products. The algae may be harvested and the oil, protein, lipids, carbohydrates and other components extracted. Organic components not utilized for biodiesel production may be recycled into animal feed, fertilizer, nutrients for algal growth, as feedstock for methane generators, or other products. The extracted oil may be processed, for example by transesterification with low molecular weight alcohols, including but not limited to methanol, to produce glycerin, fatty acid esters and other products. The fatty acid esters may be utilized for production of biodiesel. As is well known in the art, transesterification may occur via batch or continuous flow processes and may utilize various catalysts, such as metal alcoholoates, metal hydrides, metal carbonates, metal acetates, various acids or alkalies, especially sodium alkoxide or hydroxide or potassium hydroxide.

The products of the closed bioreactor system are not limited, but may include Biodiesel, Jet fuels, Spark ignition fuels, Methane, Bio-polymers (plastic), Human food products, Animal feed, Pharmaceuticals products such as vitamins and medicines, Oxygen, Waste stream mitigation (product removal), Waste gas mitigation (e.g. sequestering CO₂).

### Example 2

### Bioreactor Farming

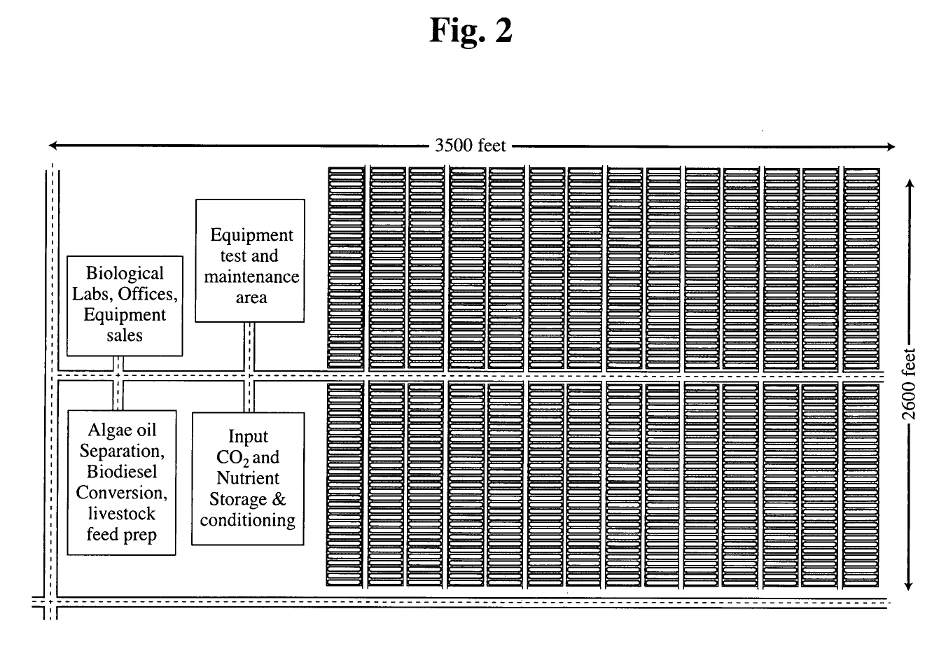

Certain exemplary embodiments are illustrated in FIG. 2, which shows an aerial view of a closed bioreactor system for algal culture. In this exemplary illustration, the algae crop is grown in substantially horizontal clear plastic tubes, laying flat on the ground, that have aqueous growing media moving through, thereby keeping the algae in suspension. (By substantially horizontal it is meant that the slope of the ground surface under a single bioreactor is level to within approximately 1 inch so that he actions of mixing, water movement, and plastic tube stress are generally consistant throughout the tube. However, the skilled artisan will realize that in other embodiments a terraced arrangement could be utilized to enable large arrays of individual bioreactors with pumping of fluid from low to high parts of the overall system.) In preferred embodiments, the tubes are thin-walled so as to be economical and are constrained by sidewalls to spread out on the ground until they are full of water about 8 to 12 inches thick. This is approximately the maximum thickness that algae laden water can be rotated through in order to expose all portions equally to red and blue photosynthetic light, which penetrates only about 1 inch because of absorption and the shading effect of other algae. The width of the tubes may be nominally about 10 to 20 feet and the length approximately 100 to 600 feet. However, the skilled artisan will realize that such dimensions are not limiting and other lengths, widths and thicknesses may be utilized. In general, nutrients, proper salinity or mineral content, CO₂, and sunlight are present in the aqueous media. The media has been seeded with a desirable algae picked to provide a particular end product and grow well in the bioreactor and so it propagates and multiplies as long as the growing conditions are sufficient. Referring to FIG. 1 Preferred System Schematic, the bioreactor is only one component of an overall system that feeds the bioreactor and harvest the algae from it.

Referring again to FIG. 2, the Figure illustrates an exemplary layout of a relatively small farm, capable of producing 6000 gallons of biodiesel a day. The view shows 1400 individual bioreactors that are connected like leaves on a fern to central servicing rails. The skilled artisan will realize that other configurations are possible, although in preferred embodiments a more or less linear bag arrangement containing the growing algae is utilized.

### Example 3

### Closed System Bioreactor Apparatus

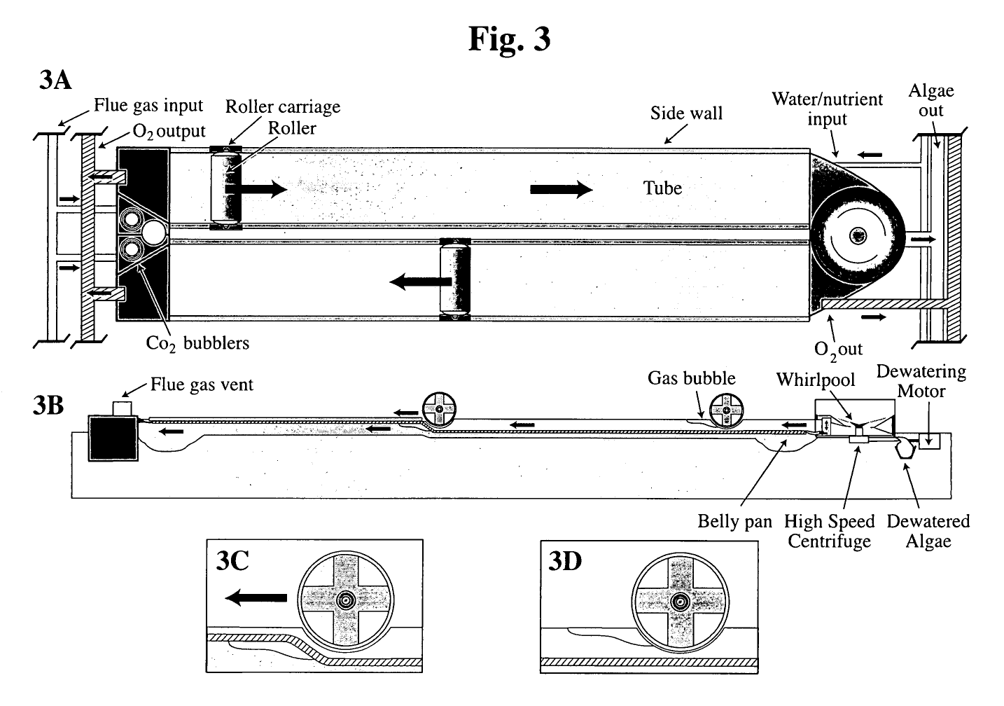

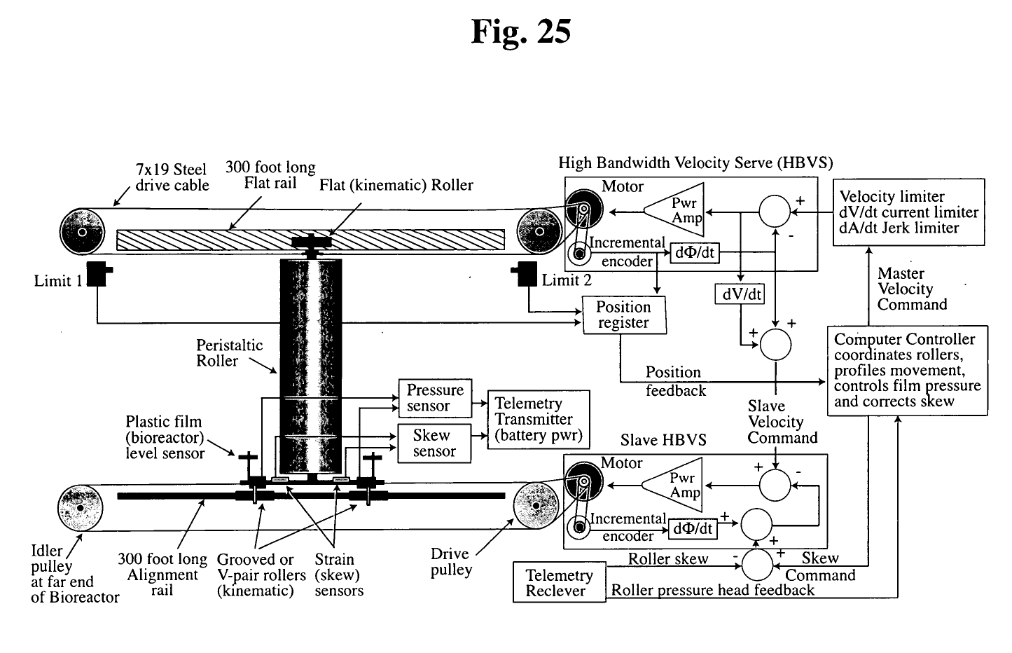

FIG. 3A-D shows a non-limiting example of a closed system bioreactor apparatus. An aqueous medium is contained in substantially transparent flexible tubes (bags), discussed in more detail below. The liquid contents of the bag may be circulated by movable rollers that roll across the surface of the bag, pushing liquid in front of them. In this non-limiting example, the rollers track along a roller support rail and are driven by cables attached to carriages that roll on the top of the rail. A roller drive system described in FIG. 25 provides a motive force for roller movement. In an alternative embodiment not shown here, when the rollers reach the end of the bag, they may be rotated or lifted upwards to travel back to the starting point in a continuous oval path. However, in the preferred embodiments shown, bidirectional rollers are used that travel from one end of a bag to the other and then reverse direction to return to the starting point, as discussed below. The use of a roller system provides liquid circulation while generating low hydrodynamic shear force, in contrast to standard mechanical pumps for fluid movement.

FIG. 3A shows an exemplary two bag system, each bag operably coupled to a roller. The bags are joined at the ends by chambers, which can hold CO₂ bubblers, a whirlpool device, various sensors (e.g., pH, dissolved O₂, conductivity, temperature), actuators for moving the thermal barrier, and connections to pipes for transport of water, nutrients and/or harvested aquatic organisms, such as algae.

As indicated in FIG. 3B, in a bidirectional roller system the tubes may be laid out along the ground, with the rollers moving substantially parallel to the ground surface. However, at the ends of the tubes, the ground under the tube may be excavated to form a dip, which may be lined with a “belly pan” as described below. This arrangement allows water in the tubes to flow under the rollers when the rollers reach the ends of the tubes and position over the belly pans. After water flow has slowed sufficiently, the rollers may reverse direction and travel back to their starting position, resulting in an alternating clockwise and counterclockwise flow of water through the apparatus.

The rollers form a kind of peristaltic pump but differ in two respects. First, the peristaltic filling force is provided by the leveling action of gravity on the fluid rather than the elastic return that is seen in many pumps. Second, the rollers only squeeze the tubes down about 85% rather than completely. This means the fluid pressure differential from front to back of the roller causes a relatively high speed reverse flow right under the roller, as discussed below. In some embodiments, the roller speed (and accordingly the fluid velocity) may be approximately 1 foot/sec.

In various embodiments, the aqueous medium may be used to culture photosynthetic algae. During photosynthesis, the algae absorb CO₂ and release oxygen gas. As the roller moves along the upper surface of the bag, oxygen, other gases, fluid medium and algae are pushed ahead of the roller. This not only moves the algae through the bag but also provides a mixing action for the medium. The rollers may push a bubble of gas in front of them. This is a combination of gases released from the water, un-absorbed CO₂, and oxygen generated by photosynthetic algae. The gas pocket in front of the rollers may be collected in end chambers and vented to the atmosphere or stored, to avoid oxygen inhibition of photosynthesis. In some embodiments stored oxygen may be reinjected into the apparatus at night to support algae metabolism during non-photosynthetic periods. Alternatively the collected oxygen may be piped to a power plant to increase the efficiency of its combustion processes. The rollers may also cause optical turnover of algae, which is desired to modulate its light input. Otherwise algae either become over-saturated with light or starved of light and the oil production goes down.

As illustrated in FIG. 3B-D, the roller does not reach all the way to the bottom of the tube. This results in a high velocity backwash, immediately under the roller, where the force applied to the liquid in front of the roller results in fluid movement backwards under the roller. This backwash has several effects, including scrubbing the bottom surface of the tube to reduce biofouling and resuspending algae or other aquatic organisms that have settled to the bottom of the bag in the medium.

A thermal barrier may be included within the bag, separating the liquid components into upper and lower layers for thermal control. Depending on how fluid movement is regulated, the liquid may be diverted primarily into the upper layer of the tube above the thermal barrier (FIG. 3D) or into the lower layer of the tube below the thermal barrier (FIG. 3C). FIG. 3B shows the rollers in two alternative positions to illustrate the septum control. When the liquid is in the upper layer, the collected gas pocket is forced against the upper surface of the flexible tube (FIG. 3D). The moving air-water interface in front of the roller then acts to scrub the upper surface of the flexible tube, reducing biofouling and maintaining light transmission of the upper tube surface. This scrubbing action may be enhanced by the inclusion of slightly buoyant scrubber disks 1 inch diameter by ¼ inch thick that are deliberately circulated in the fluid and that tend to be pushed ahead of the roller. Other solid shapes of similar size may be designed by those skilled in the art of scrubbing the inside of fluid systems. In practice, thousand of these disks or other solid shapes would be resident in the bioreactor but not so many as to reduce the light transmission appreciably. They would be separated from the algae mixture with screens before harvesting and would be sufficiently low buoyancy that they could be washed into the air bubble space ahead of a roller by the prevailing fluid current caused by the previous roller. When the liquid is in the lower layer (FIG. 3C) the underside of the thermal barrier layer is scrubbed in the same manner to maintain light transmission through it.

As shown in FIG. 3A-B, mechanisms may be incorporated into the apparatus, for example at the ends of the bag, to harvest algae, add or remove gases, nutrients and/or waste products or for other purposes. In a preferred embodiment, the hydrodynamic fluid movement at the ends of the bags may be designed to promote formation of standing whirlpool circulation, discussed in more detail below, which may be utilized to improve efficiency of aquatic organism harvesting, gas and/or nutrient introduction, waste removal, or for other purposes. The right side of FIG. 3A-B shows a whirlpool device for harvesting aquatic organisms, discussed in more detail below.

The illustrative embodiment shows a research model that is only 65 feet long, with individual bioreactor bags that are 52 inches wide. In a preferred production scale embodiment each of the two bags would be about 300 feet long and 10 to 20 feet wide for a total photosynthesis area of 0.15 to 0.30 acre per bioreactor assembly. Each such bioreactor should grow about 7 to 14 gallons of biodiesel per day or more.

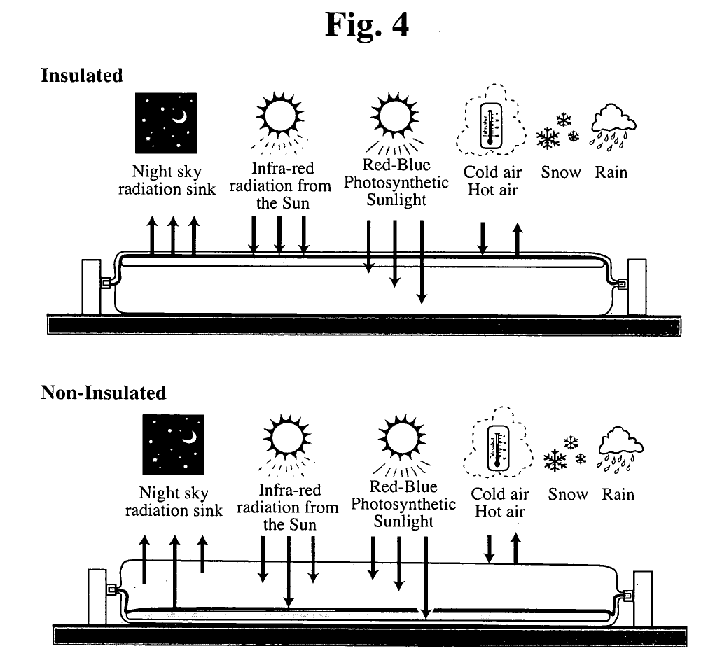

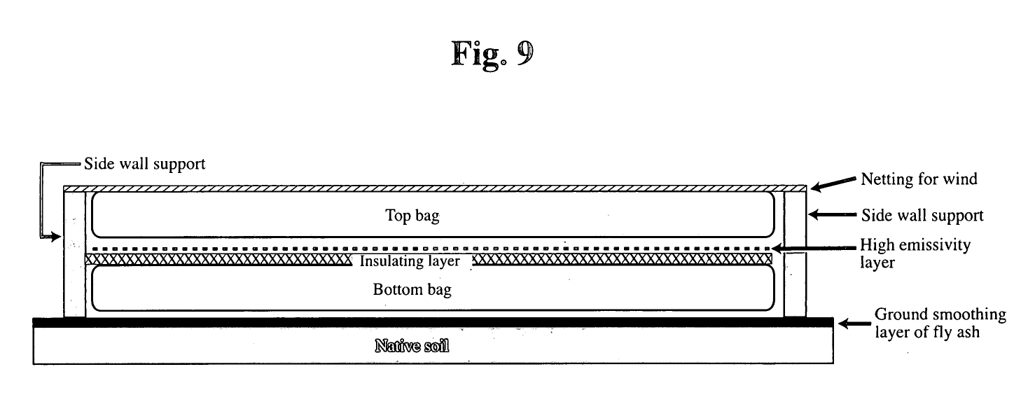

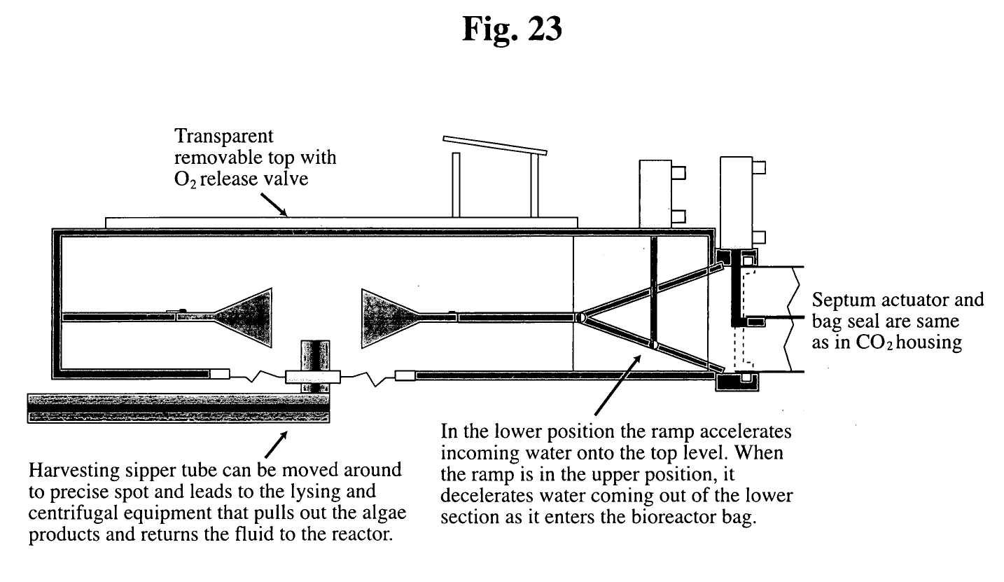

In some embodiments, a single tube may be formed to contain an upper layer, internal thermal barrier, and lower layer as shown in FIG. 4 and on the right side of FIG. 23. In alternative embodiments disclosed in FIG. 9, a dual bag system may be utilized with separate upper and lower bags and a thermal barrier in between. In operation, such a system would behave identically to the single bag system discussed above. The advantage of the dual bag system is that it potentially eliminates the need for sealed side seams, providing greater structural stability and decreasing costs. Further, since the high emissivity layer and insulator (discussed below) do not need to be waterproof, there are additional options for selection of materials. Also, since the thermal barrier layer is not exposed to the algae, it eliminates the possibility of biofouling of that material. Finally, the insulator and high emissivity layer may be retained when the bags are replaced, providing additional cost savings. FIG. 9 also shows an optional layer of a ground smoothing layer, such as fly ash, deposited between the bag and the ground, which may be used with either a one-bag or two-bag system. Fly ash is a low cost material that may be obtained in the local of power plants and one that has a sufficient caustic nature as to retard the growth of plants under the bioreactor bags. Other materials including salt may be placed under the bags to retard growth. A netting over the top bag is optional.

### Example 4

### Thermal Control of Aqueous Medium

In the exemplary embodiment of FIG. 3, the tube in a preferred configuration has a construction that includes a high emissivity insulating septum (thermal barrier) installed horizontally down the center. The last few inches of this septum may be stiffened with a bar that can be driven up by actuators to close off the upper tube, or down to close off the lower tube. The bar is constructed with a flexible sealing lip that serves as a one-way valve permitting fluid or gas flow out of the upper or lower tube even when the septum is clamped to prevent fluid entry. This permits the roller to squeeze out residual fluid or gas from a chamber regardless of septum valve position. The left hand roller (FIG. 3C) appears to be rolling the fluid in the bottom of the tube, below the thermal barrier, out into the left hand chamber. After that fluid recirculates back around to the right side, where the septum is in the down position, it is channeled above the thermal barrier, allowing the fluid to fill the top of the tube. This is an example of how the septum position can cause the movement of fluid between the upper and lower parts of the tube without much energy usage. The purpose of this movement is thermal control of the fluid.

A non-limiting example of bioreactor thermal control is illustrated in FIG. 4, which shows a cross section of one flexible tube looking through it lengthwise. The purpose of thermal control is to keep algae in the medium at their optimum temperature and prevent the tubes from freezing at sub-zero ambient temperatures, or from overheating during hot summer days. The thermal control aspects involve use of different bag components with selected optical and/or thermal transmittance properties. For example, a top sheet (e.g., 0.01 inch thick clear polyethylene) may allow light in and heat in or out. An internal thermal barrier may comprise a flexible sheet that is designed to absorb infrared but pass visible light for photosynthesis, overlaying a conductive insulator. In some embodiments, the thermal barrier may be a composite comprising a flexible insulator sheet bonded to an IR absorbing sheet. The insulator may comprise, for example, a ½ inch (R2) or 1 inch (R4) thick layer of foamed polyethylene. The tube also comprises a bottom sheet that is normally, but not necessarily, identical in composition to the top sheet.

The tube may be formed by side sealing two sheets (upper and lower) or three sheets (upper, thermal barrier, and lower) of flexible plastic, although other mechanisms may be utilized, such as providing a seamless tube by continuous extrusion or blowing of a cylindrical sheet of plastic. A ground sheet that is resistant to physical/mechanical disruption but is heat conductive may be placed between the ground and the tube. The ground may be treated or prepared to be relatively flat, smooth, heat conductive and plant resistant. Side walls may be provided to physically support the fluid-filled tube and/or provide additional thermal insulation from the sides of the tube and additionally to support and guide the roller carriages.

As shown in FIG. 4, in a non-insulating mode, water is channeled above the thermal barrier in the tube, allowing heat emission to cold (night-time) air or heat absorption from solar infrared radiation during the day. This mode also allows maximal absorption of visible light for photosynthesis. Heat transfer may also occur by conduction or convection as well as IR emission or absorption. In insulating mode, the fluid is channeled below the thermal barrier, thermally stabilizing the fluid temperature by contact with the thermal mass of the ground. The thermal barrier insulates the fluid from solar IR radiation. Visible light may still pass through the thermal barrier to support photosynthesis, although the efficiency of transmission is less than 100%. During the night, ground contact would warm the fluid, while during the day, ground contact would cool the fluid. In some embodiments, heat transfer to or from the ground may be used to pump the ground as a thermal sink or source for use in moderating the fluid temperature during the day or night. For example, transferring heat to the ground during the day and absorbing it at night to keep the fluid warmer in winter months or transferring heat from the ground during the night and using the ground as a heat sink to cool the fluid during the day in the summer.

In alternative embodiments, active thermal control with power plant water may be utilized. Heated water from a power plant's cooling towers may be pumped to a plastic mat placed under part of the bioreactor tubing. When it is cold this additional heat source may be utilized to prevent freezing and/or below optimum algal growth temperatures. The skilled artisan will realize that a variety of heat sources may be utilized, such as power plant exhaust, geothermal heat, stored solar heat or other alternatives. Additionally in hot seasons or locations of high solar flux, evaporative or other cooling systems that can be efficiently powered can be used to keep the algae from overheating.

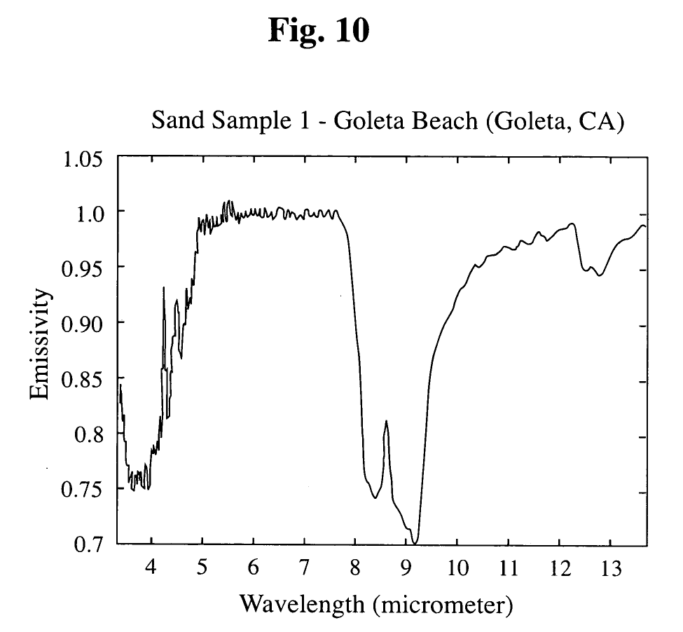

In some embodiments, the emissivity properties of the thermal barrier may be adjusted by incorporation of other materials of selected optical characteristics, such as quartz sand (ee, e.g., FIG. 10), doped glass or quartz beads or tiles of selected optical properties that might be embedded within the upper surface of the thermal barrier.

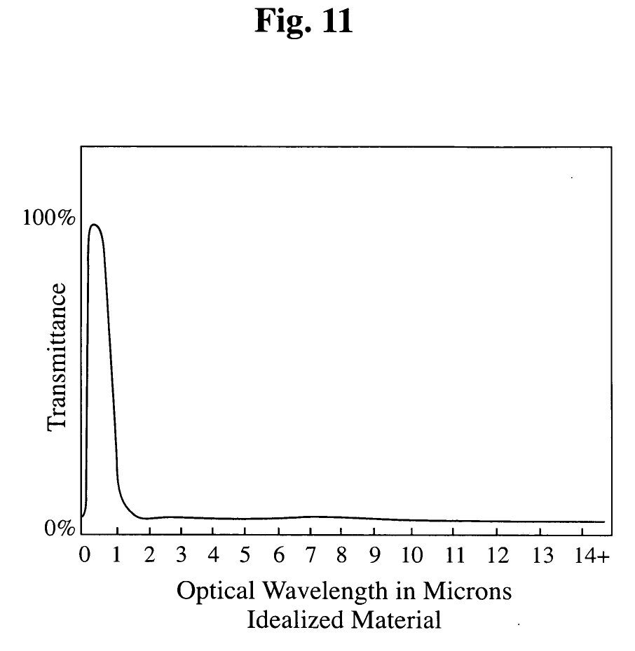

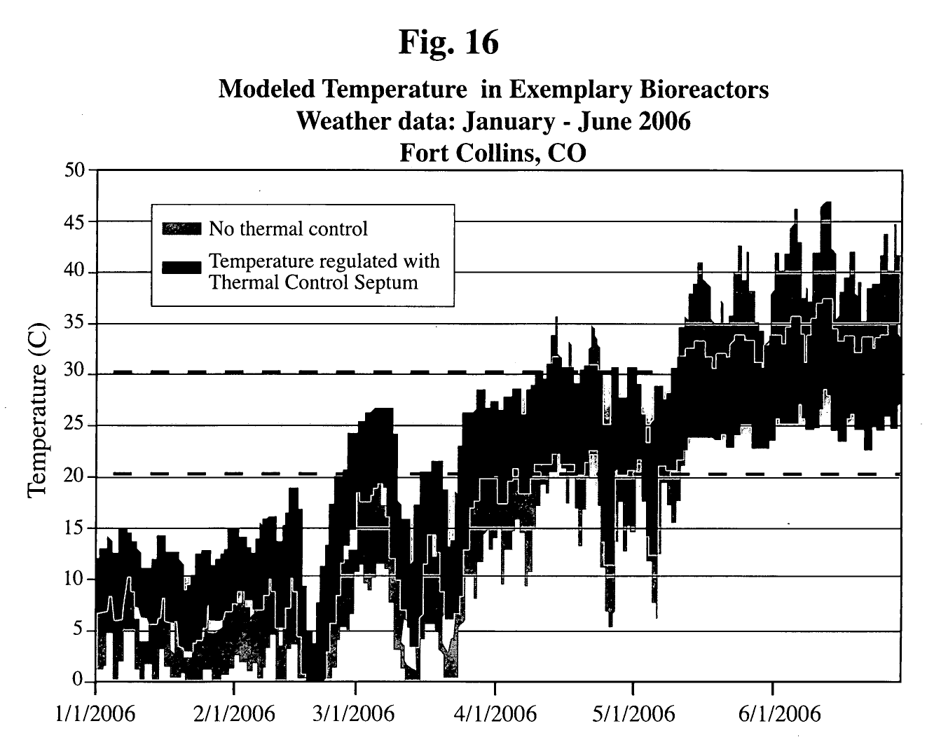

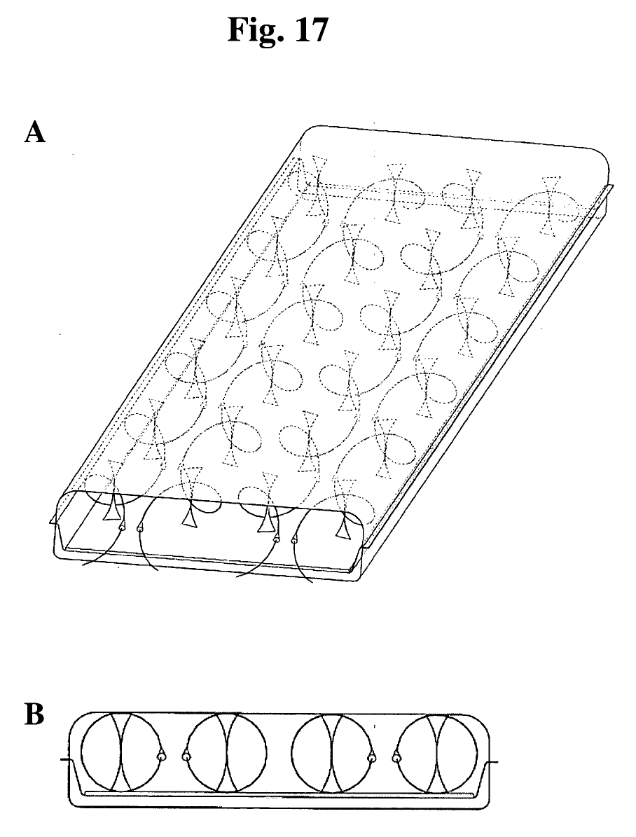

The thermal control mechanism discussed above is highly effective at maintaining temperatures in a range for optimal algal growth. FIG. 16 shows computer modeled water temperature data, using the environmental conditions at Fort Collins, Colorado between January and June, 2006, with an R-4 (1 inch thick foam) thermal barrier and an ideal infrared absorption layer (see FIG. 11). The water temperature ranges are modeled with (gray) and without (black) the presence of a thermal barrier. It can be seen that Spring and Summer temperatures were largely stabilized in the range of 20 to 30° C. with the thermal barrier, whereas in the absence of the thermal barrier the summer water temperature reaches 45° C. or higher. The thermal barrier decreases maximum summer temperature by about 10° C. The barrier is less effective at maintaining winter water temperature in the optimum range. Various alternatives are available for winter aquatic organism production, such as use of heat from supplemental sources (e.g., power plant exhaust), location of production units in warmer climates where winter temperature is not as cold, or use of cold-tolerant algal species such as Haematococcus sp.

### Example 5

### Whirlpool and Sipper

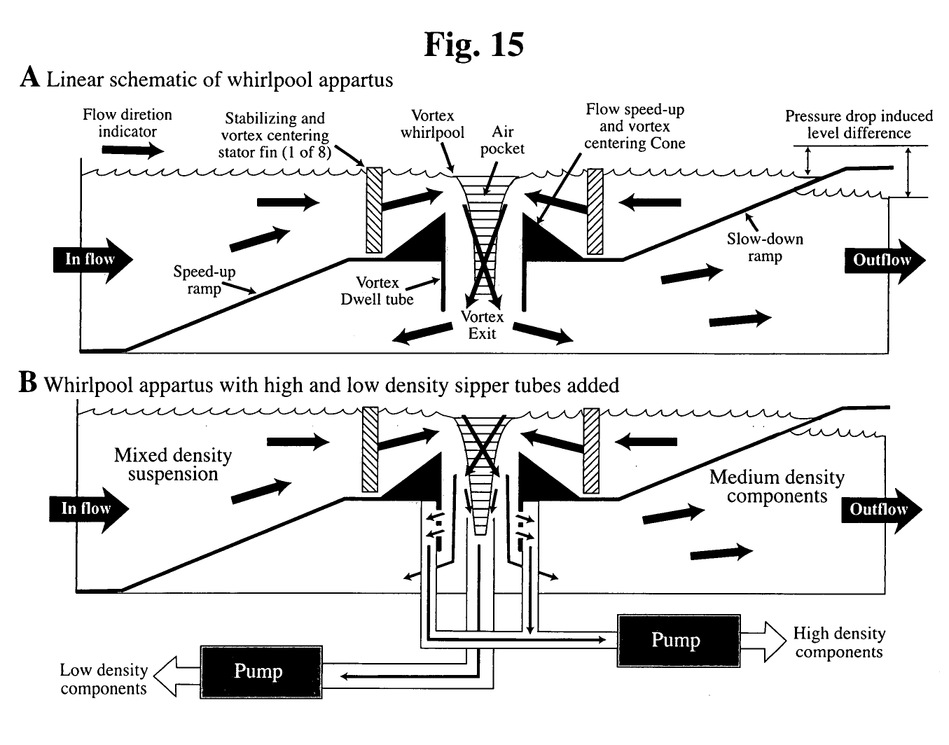

An exemplary harvesting whirlpool of alternative design is illustrated at the right side of FIG. 3 and the preferred dwell tube design is shown in detail in FIGS. 15A and 15B. Although preferred embodiments of a bioreactor include such a whirlpool device, the apparatus is not so limited and in alternative embodiments other methods and devices for harvesting algae from the medium may be utilized. The primary purpose of the whirlpool is to permit extraction of fluid which is enhanced with algae (or other aquatic organisms) containing a desired product. A secondary purpose may be to extract components of the fluid that need to be removed from the medium, like mucilage or foam that may primarily consist of deleterious bacteria. There are numerous potential uses for a density separating whirlpool, corresponding to the many different product types that may be grown in a photo-bioreactor. Algae of different species and in different environmental circumstances or life stages may be either heaver or lighter than the fluid medium, depending upon their concentration of oil, carbohydrates, and gas vacuoles, as well as the growing media that can have various densities depending on salt content and temperature. Aquatic organisms other than algae may also be separated from the liquid via density differences in this manner.

As shown in FIG. 15, as fluid leaves the tube septum valve area (marked IN FLOW) on the left it is crowded up onto a ramp positioned at the ½ depth position and is consequentially speeded up by a factor of approximately 2. The fluid may then surround and impinge against a speedup cone and then flow over its edge and drop through a dwell tube into the bottom of the chamber. The drop into the dwell tube induces a whirlpool vortex action, with the fluid spinning faster and faster as it enters the hole. How fast it spins, and the degree of centrifigual force resulting from the whirlpool is proportional to the ratio between the hole area and the bag cross-sectional area as well as the roller speed and tube squeeze ratio. The purpose of the dwell tube is to maintain the centrifigual separation forces for as long a dwell time as possible before the liquid must de-spin into the lower chamber. As the heavy salt or mineral laden water and heavy or flocculated algae is pushed out towards the outside of the spinning whirlpool in the dwell tube, the gas bubbles, lower density algae, and other low density components migrate to the center of the whirlpool. A “sipper” tube may be positioned at the center of the whirlpool (FIG. 15B), optionally with a variable diameter aperture, to collect the central contents of the whirlpool which may be enriched in a particular product. The sipper de-rotates the mix and feeds it into a screw-drive dewatering filter, or high speed continuous centrifuge, or both, or other extraction and dewatering devices. The nutrient containing water after product removal may be filtered to remove residual biological fragments that might support bacterial growth, then sterilized with UV light and returned to the bioreactor. The dewatering device may transfer the condensed algae or other product to a collection conveyor belt or other apparatus to collect the algae from many bioreactors arranged in a line and to deliver large quantities to a central processing facility for oil extraction. The algae may partition into clumps and drop through space as it lands on the conveyor line, or may be channeled through bioseptic one-way valves to prevent the possibility of a foreign organism on the conveyor line entering the bioreactor and causing a disruption or “infection” of the monoculture to spread from one reactor to another. In another configuration, also shown in FIG. 15B, the sipper may consist of perforations on the inside of the dwell tube to collect the highest density components of the fluid. These, for example, may be algae rich in both oil and carbohydrates in a proportion that makes the algae heavier than the medium.

Another purpose of the whirlpool may be to serve as an alternative CO₂ injection mechanism. This would happen on the bottom of the whirlpool where the fluid is spinning outward after leaving the control orifice. Gases like pure CO₂, or alternatively CO₂ rich flue gases obtained from a power plant, factory or other source, may be injected mid radius in the vortex or just below the opening of a central sipper tube. In this position the bubbles are prevented from seeking the center of the vortex because of the restriction caused by the sipper tube and the downward counter flow of the water. Yet because the force of buoyancy and downward flow are concurrently present, there is a dwell time until the bubble blows large enough from its source orifice. Its size constricts and speeds up the water flow around it so that the bubbles are sheered off the generating orifice as small bubbles that are carried in the slower flow. In preferred embodiments, much of the gas is absorbed into the fluid before the bubbles coalesce and rise to the top of the tube.

It may be possible for the bioreactor to aquire CO₂ directly from the air either by bubbling up air through neoprene injectors or by direct permeation through the top skin of the bioreactor. In some embodiments, on the top inside of the tube there may be deposited 1 inch diameter pockets of sodium hydroxide mixture, sealed behind a gas permeable but water proof membrane, perhaps composed of a polystyrene membrane which has been shown to be very permeable to CO₂. As these pockets are partially exposed to the outside atmosphere, they can selectively absorb the CO₂ component of air. Then as the roller passes over the pockets they are physically compressed by the roller such that the top is sealed and the partial pressure of the CO₂ is higher than in the water on the bottom side of the membrane and rapid transmembrane diffusion occurs into the liquid. In this construction the top sheet looks a bit like bubble wrap with the bubbles on top and filled with a sodium hydroxide mixture and both the bottom and top comprising CO₂ permeable membranes. In an additional embodiment for direct CO₂ acquisition, the top skin of the bioreactor is made of a composite of open-celled fabric as a strength component with the pores filled with a CO₂ permeable and absorbing substance. This may be polystyrene microcapsules of sodium hydroxide. In operation the capsules would absorb CO₂ from the air then either dispense the CO₂ directly to the fluid through passive diffusion or through pressurized diffusion when the roller compresses the capsules on each sweep.

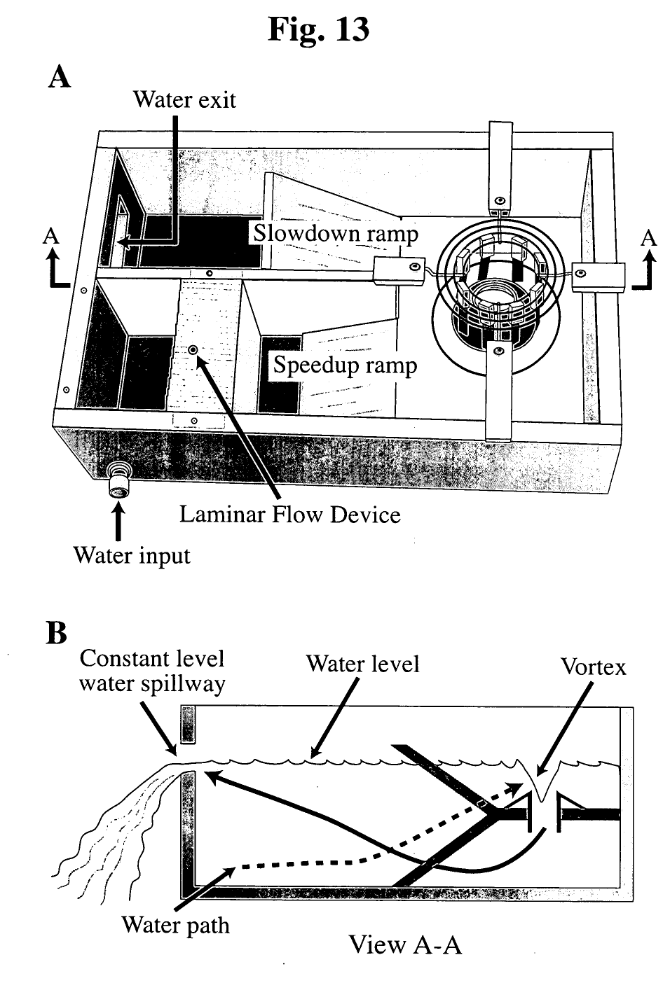

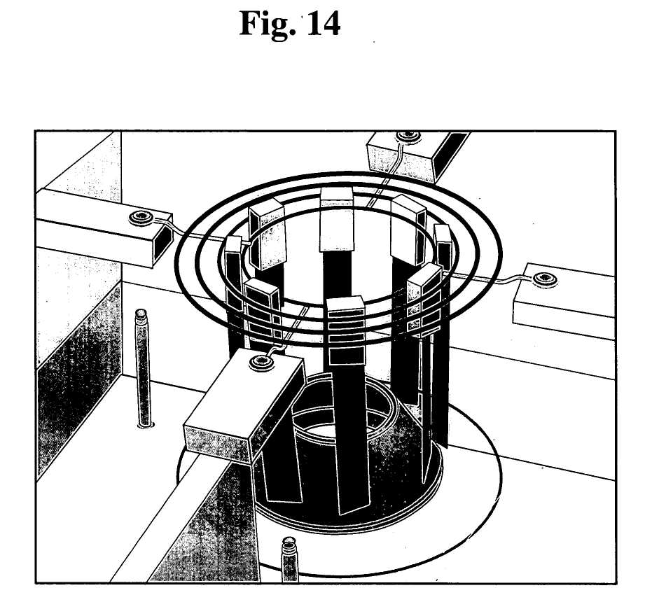

An exemplary model of a whirlpool device is shown in FIG. 13. Water enters a chamber, such as a first control housing, and encounters a speed up ramp that accelerates the water velocity and moves the water on top of a deck positioned midway in the total fluid depth. The water further accelerates up over the speedup cone and drains down through a dwell tube where the whirlpool naturally occurs. Water exiting the bottom of the dwell tube enters the chamber below the central deck and flows outwards through an upward sloping slowdown ramp before exiting the control housing. The purpose of the ramps is to gradually change the speed of the water flow to prevent whirlpool disruptive turbulence as it flows onto the top of the mid-deck or out from underneath. Details of the dwell tube and speed up cone are shown in FIG. 14. As discussed above, water descending to a lower level through a constriction naturally forms a whirlpool, much like a toilet being flushed. The dwell tube, speed up cone and stator fins discussed below are designed to facilitate formation of and stabilize the whirlpool at the center of the dwell tube. The length of the dwell tube is designed to increase the dwell time that the liquid suspension is under centripetal force, maximizing separation of different density components such as the lighter or heavier product-filled algae and the water medium. Stator fins surrounding the dwell tube provide a centering force that stabilizes the position of the whirlpool in the center of the dwell tube. This may be important because the sipper apparatus may need to be precisely positioned within the whirlpool to sip only a thin ⅛″ layer of speeding water. The stablizing stator fins act as a turbulence filter around the whirlpool. Because of their angle, side to side sloshing in the control housing is damped from disrupting the vortex position, while spiral motion of the entering water is unimpeded. Under experimental conditions, the model whirlpool device shown in FIGS. 13-14 formed a stable whirlpool.

The fluid mechanics of the whirlpool device are illustrated in FIG. 15A. Water flowing into the chamber encounters a speed up ramp and cone, centered over a hole that allows fluid descent to a lower level. This results in whirlpool formation. The whirlpool is stabilized in position by the whirlpool centering stator fins. Fluid exits at the bottom of the whirlpool and encounters a slow down ramp before exiting the chamber, resulting in relatively constant influx and efflux rates from the chamber. In certain embodiments (FIG. 15B), sipper tubes and pumps may be used to remove low density components (e.g., oil filled algae) or high density components (e.g., algae filled with carbohydrate). Although the exemplary whirlpool device is illustrated with a unidirectional fluid flow, in alternative embodiments the positions of the speed-up and slow-down ramps may be adjusted so that whirlpools may form with fluid flowing in either direction, as with a bidirectional roller system.

The purpose of the speed-up ramp and cone is to minimize turbulence as the fluid is speeded up for entry into the whirlpool, where it further speeds up in its spiral motion to provide centripetal force. It is estimated that the apparatus shown in FIGS. 13-15 would only dissipate 50 watts of power from turbulence in a full scale system capable of delivering 90 gals/sec through the whirlpool. Various alternatives exist to separate algae from the medium, as discussed above, and any such known methods may be used.

### Example 6

### CO₂ Uptake

In certain embodiments, exhaust gases that are enriched in CO₂ may be utilized to support photosynthetic carbon fixation, while simultaneously scrubbing the exhaust gases of their CO₂ content to prevent further buildup of greenhouse gases. In this way huge amounts of, for example, power plant flue gases can be “mined” for their CO₂ and the resulting gas piped to the algae farm.

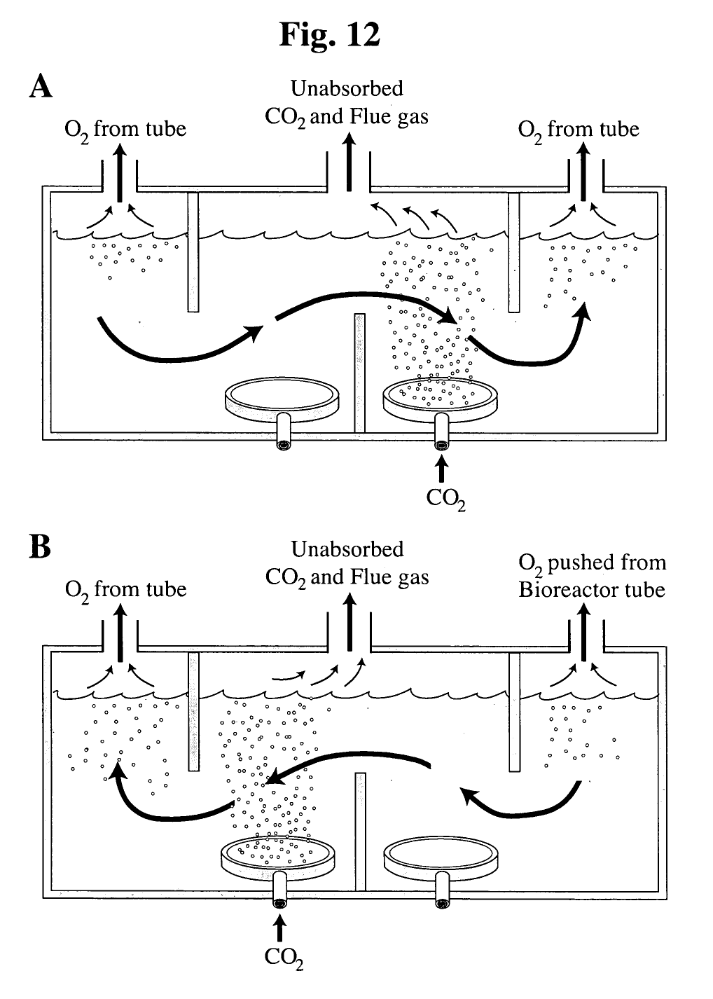

FIG. 12 illustrates an exemplary embodiment of a mechanism for CO₂ dissolution. The Figure shows a bubble generator, for example a neoprene membrane pierced with a multiplicity of small holes, located at the bottom of a water column. The bubbler generates a large number of very small diameter bubbles to promote dissolution of the CO₂ gas in the medium. While the bubbles move up due to buoyant density, the water column moves down due to the directional flow induced by rollers or other fluid transport mechanisms. The counterflow prolongs the dwell time of bubbles in the medium and maximizes gas dissolution. The length of the water column may be increased to further promote gas dissolution. In an exemplary bidirection flow system, as discussed below, where the fluid alternately moves in opposite directions, two gas bubblers located on either side of a central partition may be utilized so that the counterflow mechanism may be utilized with either direction of fluid movement (FIG. 12A, FIG. 12B). In this configuration, CO₂-containing flue gas may be piped for miles from a power plant to the bioreactor farm. Mathematical modeling of this process indicates that it would be a sufficiently energy efficient process to pipe CO₂ to the bioreactor and to remove CO₂ from flue gas in the bioreactor.

Where long flexible tubes are used, it may be optimal to provide a supplemental CO₂ injection mechanism at both ends of the tube. It is estimated that aquatic organisms flowing at 0.25 meter/second would require additional CO₂ approximately every 7 minutes (105 meters). Supplemental CO₂ could be provided in a variety of forms, such as gas bubbles, water pre-saturated with CO₂, addition of solid forms of CO₂ (e.g., NaHCO₃, Na₂CO₃, etc.)

### Example 7

### Roller Drive

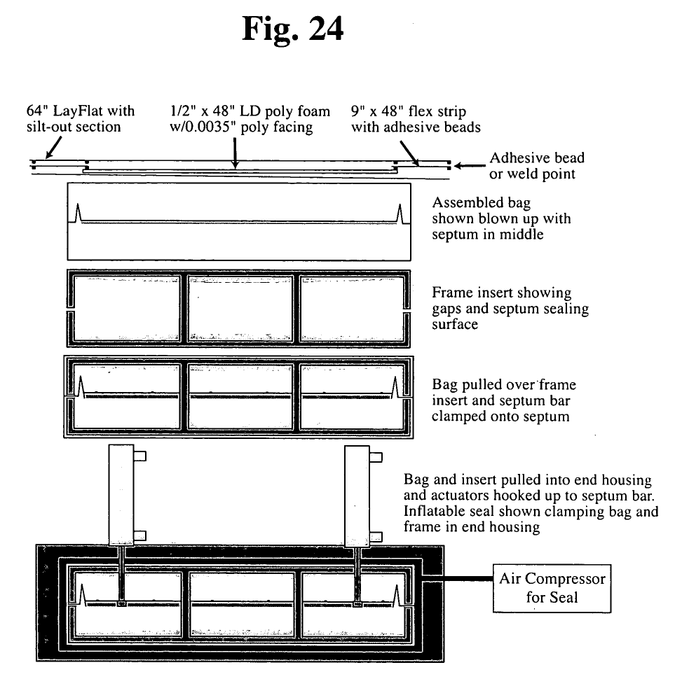

FIG. 24 shows a preferred roller drive system. The rollers may be thin and lightweight tubes, for example of fiber glass and fiber construction. Alternatively, the rollers may be stainless steel or other heavy cylinders. In either case they must be heavy enough to compensate for the volume of water they displace underneath themselves. In most cases this will be achieved by manufacturing a thin light weight cylinder that can be inexpensively manufactured and transported and then filling it with sufficient water, sand or other material to give it the proper weight after installation. The rollers may comprise a solid axle between two support roller assemblies. In a preferred version the rollers are either independently driven on each side or there is a driven differential mechanism between them. This is because the roller perpendicularity to the drive direction is critical to prevent bunching or wrinkling of the bag assemblies. Sensors may detect when one side of a roller is getting ahead of the other or when cross track is being put on the bags and adjust the phasing of the drive from one side to the other so that the rollers smoothly track over the bags with out causing damage or incurring excess friction. The kinematic design of the roller carriage system in FIG. 25 permits it to compensate for large misalignments and temperature changes.

Ten to twenty foot long rollers must be accurately driven, against a background of reflected waves, misalignments, temperature differences, and varying friction in order to avoid skewing of the roller and diagonal wrinkling of the tube. In certain embodiments, the rollers may weigh thousands of pounds and may move along a track that can be 300 feet or greater in length. The exemplary system shown in FIG. 25 utilizes a steel drive cable system, which is low cost and has low driveline inertia because the cable transmits force through tensile strength, which is very mass efficient. In this embodiment, nested, high bandwith velocity servos are used to drive the drive pulleys and keep the rollers from skewing.

The velocity command of the upper master servo is derived from the controller by determining the difference between where the roller is and where it should be. By limiting the first and second derivatives of the resultant velocity command, the unstable water filled bioreactor bags are minimally excited. Wave action oscillation from any source is not magnified and does not induce out-of-phase feedback signals due to drivetrain compliance, because the velocity feedback sensors being directly attached to the drive motors are isolated from compliant elements. The bottom servo is slaved to match the same velocity as the upper main servo but with enhanced velocity following due to the dV/dt lead feed-forward network in its command. The slave velocity command is summed and offset by the skew strain sensor outputs on the kinematic carriage system. This actively drives the roller to a precise angular alignment referenced to the alignment rail. The exact angle of skew can be adjusted by the controller to compensate for roller directionally unique effects or to relieve detected wrinkle formation in the bioreactors. The controller can also use the fore-aft roller hydrostatic pressure difference sensed by the film (bioreactor tube) level sensors to control the roller velocity in order to maintain a specific pressure head. Battery or solar powered skew and level sensors with RF telemetry output require no power wires to be hooked to the roller. The carriage system is of kinematic mechanical design. This provides that changes in width between the roller rails or roller length changes due to expansion do not bind the carriage system. It also means that the roller perpenducularity is constrained by only one carriage end and therefore can accurately be measured by sensors on that end and the result used to differentially control the drive systems velocity on each end so as to zero out accumulated skew.

### Example 8

### Tube Coatings

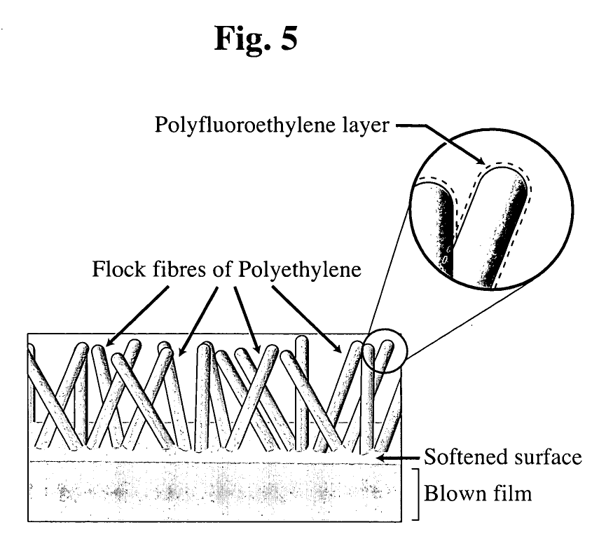

Technology for preventing or delaying bio-fouling of the inner plastic layers by algae adhering is important. This is because if the bags need to be replaced too often then it becomes an economic drain on the operation. There are a number of approaches to preventing biofouling under development worldwide although nano textured hydrophobic surfaces that are very pointy on a nano scale are one possibility. (See www.awi-bremerhaven.de/TT/antifouling/index-e.html). One way to make a non-fouling inner surface for the bioreactors at very low cost is to use flocking technology to electrostatically embed the ends of polyethylene fibers that are approximately 1-2 microns diameter by 10-20 microns long into the soft, still cooling, polyethylene plastic blown film “bubble” just as it leaves the blown film annular nozzle. (See e.g. www.bpf.co.uk/bpfindustry/process_plastics_blown_film.cfm to understand the blown film process. See e.g. www.swicofil.com/flock.html for details regarding flocking.) A non-limiting example of a flocking based substrate is illustrated in FIG. 5. Alternatively a tacky or curable adhesive coating may be applied to the inside of the tube or to one side of a sheet of plastic film used for tube construction prior to the flocking of the fibers and exposure to fluorine gas.

The inner flocked surface on the inside of the bubble may be made hydrophobic by having the inside of the bubble pressurized with fluorine gas (rather than air) which reacts with the polyethylene to create a thin skin of hydrophobic polyfluoroethylene (which is similar to polytetrafluoroethylene, PTFE) on both the flock fiber's surface as well as the plastic film between the fiber bases.

In certain embodiments, the bag may be made completely black on at least one side of the two bag system. When algae goes into the darkness it consumes oxygen and when in the light it produces oxygen. There may be an oil productivity advantage if even during the day the algae mixture is channeled alternately through light and through darkness on some selectable duty cycle so as to consume some of the dissolved oxygen in the fluid and stimulate the energy converting photosynthesis reactions.

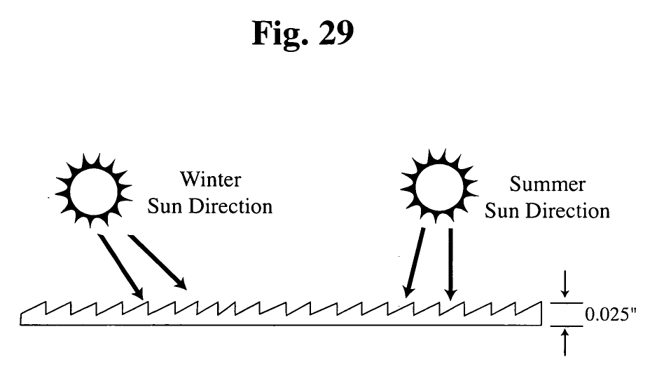

In various embodiments, the top surface of the tube may be patterned to maximize light absorption for photosynthesis during the winter months, particularly at higher latitudes. An exemplary Frenel pattern is shown in FIG. 29, which illustrates a cross-section of the tube's top layer, with Frenel light gathering prisms that are oriented east-west with the angled face pointed towards the equator. The overall thickness is 0.025 inches and the Frenel pattern is created during the plastic blowing process or during a post rolling process.

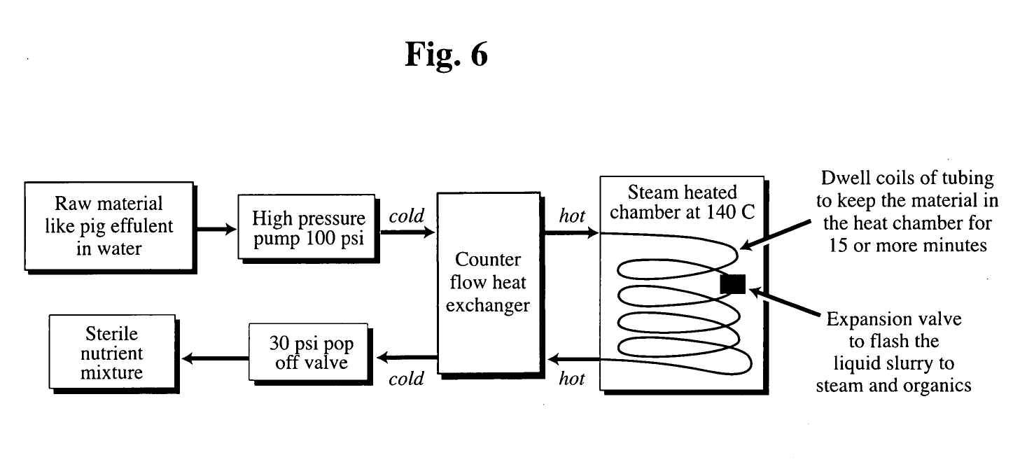

Everything that goes into the bioreactors is preferably sterile except for the desired seed culture of the microorganism. In order to do this inexpensively on an industrial basis we may utilize a continuous flow autoclave (FIG. 6). This may be done not only for the nutrients but also for any liquid returned to the bioreactors. Gases like air going into the bioreactors can be HEPA filtered and smokestack gases can be assumed to be sterile from the power plant heat. Return fluids which are optically clear may be sterilized using UV light technology.

### Example 9

### Oil Extraction

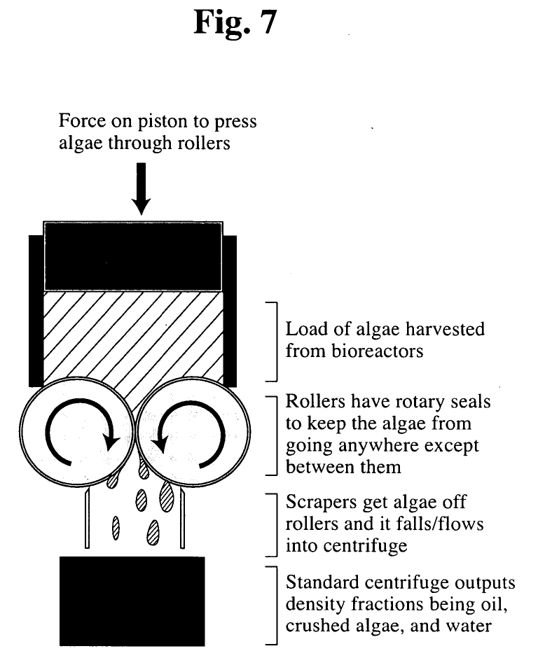

An exemplary method and apparatus for oil extraction and/or centrifugation is illustrated in FIG. 7. Algae may be extracted and their oil product removed without complex chemical treatment. The simplest way for large algae is to crush the algae and centrifically separate the components into oil, crushed algae bodies for feed or nutrient, and nutrient laden water. However, algae is slippery and may be difficult to crush by standard means. FIG. 7 shows a non-limiting example of algal crushing and oil extraction. The two rollers may be made of different materials. One may be a ground cylinder of hardened metal similar to a printing press roller. The other may be an accurate metal cylinder with a compliant rubberized coating about 0.25 mm thick. The coating makes up for small imprefections in the roller surfaces, allows small grains of sand to pass, yet provides sufficient localised pressure to burst algae bodies. Alternative harvesting methods may use various versions of rotating and vibrating screen technology to remove the largest organisms. There are many machines used for this purpose in the manure handling industry and they may be adapted by miniaturization and made economical so each bioreactor has one. This is useful because anything dipped in one bioreactor should not be dipped in another in order to avoid potentially spreading infection. Ideally, as algae is harvested by a mechanism attached to each individual reactor then the resultant water can be filtered of residual organic material and then directly injected back into the same reactor without re-sterilization.

### Example 10

### Remote Sensing

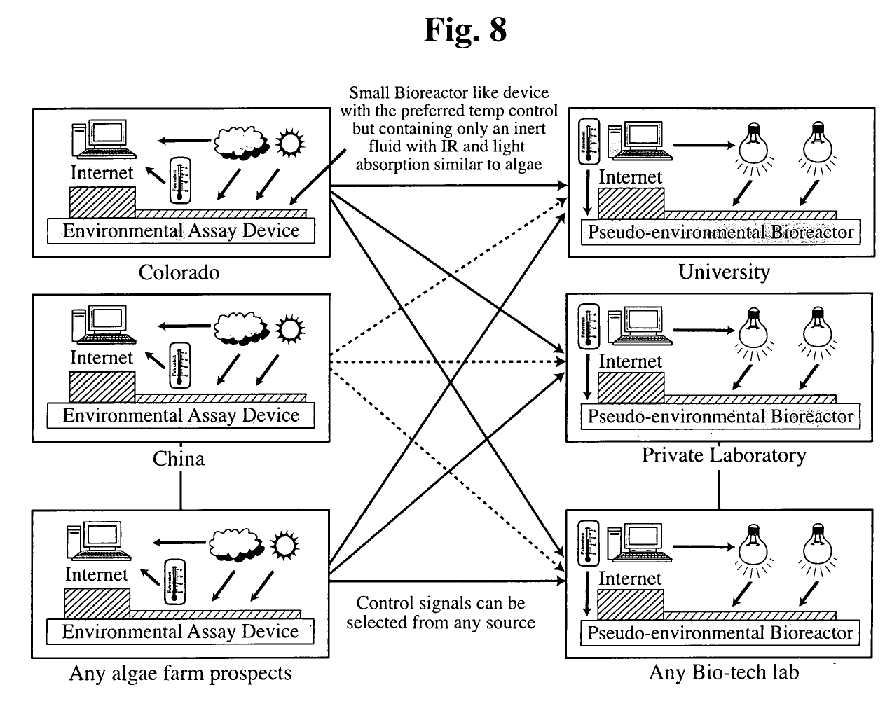

An example of a remote sensing bioreactor for condition optimization and algal strain selection is shown in FIG. 8. The system uses sensors on remote pseudo reactors that operably respond to local environmental conditions at a variety of geographic locations where bioreactors may be installed. The pseudo reactors are small bioreactor-like devices that contain an inert fluid with IR absorption and light absorbing capacities similar to a dense algal culture. The sensors detect the resulting temperatures that the pseudo-reactors are able to stabilize to as well as the photosynthetic light falling upon them. The remote sensing stations may be used to drive the temperature and light conditions of small experimental reactors in biotechnology labs so the remote environments may be duplicated in the lab for convenient strain selection. The remote environmental assay device is designed to mimic the response of a bioreactor in situ. This is more accurate than a sensor-only system since the environmental assay device is exposed to all the environmental variable factors that would affect bioreactor function and the input is reduced to an equivalent light exposure and fluid temperature for the pseudo-environmental bioreactor.

In another exemplary, sensor-only based embodiment, one or more environmental monitoring stations may be located to monitor environmental conditions, such as temperature, ground thermal conductivity, ground thermal capacity, humidity, precipitation, solar irradiation, wind speed, etc. The detected conditions may be transmitted to a laboratory based test bioreactor apparatus, where the test site environmental conditions may be replicated in a controlled setting.

In either embodiment, various strains of aquatic organisms (e.g., algae) may be inoculated into the test bioreactor apparatus and their growth and productivity monitored. Strains selected for optimal growth and/or productivity at any desired production location may be determined at minimal expense and maximal efficiency.

### Example 11

### Algal Culture in Model Bioreactor System

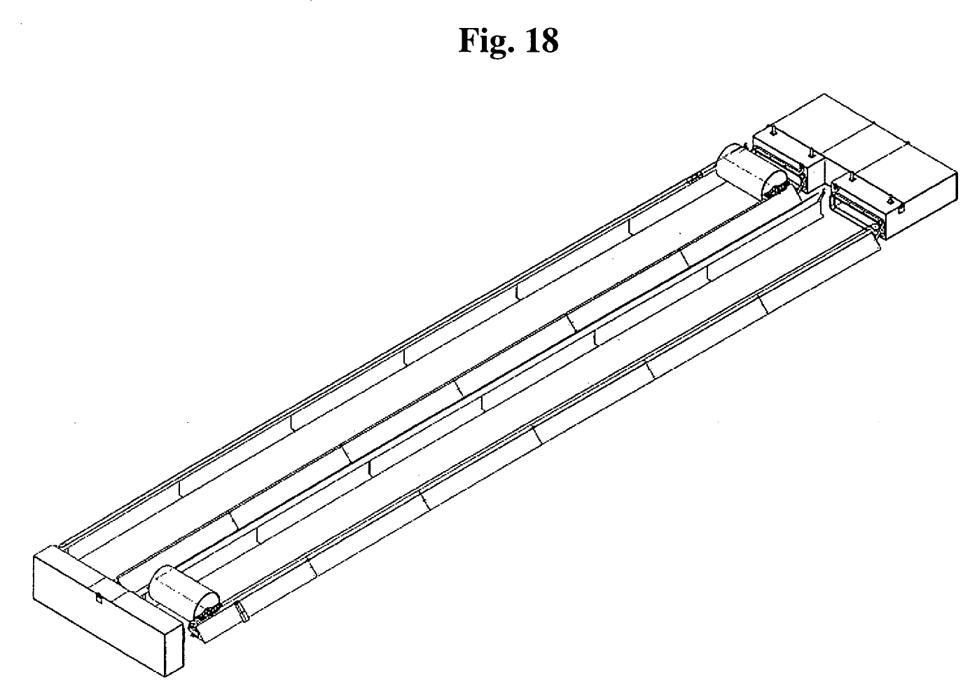

A ⅕ scale model closed system bioreactor was constructed as shown in FIG. 18. The flexible bioreactor tubes are not shown for clarity but lie in-between the two sets of guard rails and are of the same height. On the lower left is the CO2 injection housing and on the upper right is the harvester housing. The flexible tubes were constructed as shown in the top two images of FIG. 24 from two layers of 0.01 inch thick polyethylene, with a 0.5 inch thick polyethylene thermal barrier assembly layer (Sealed Air Corp., Elmwood Park, N.J.) inserted between. The three layers were sealed together by thermal impulse bonding, using a short heated bar and applying mechanical pressure. However, the skilled artisan is aware that other alternatives for thermally sealing plastic sheets, such as hot air sealing, may be utilized. To avoid shrinkage, stabilizing fibers may be embedded in or attached to the plastic sheet so that the tube geometry is not deformed by hot air sealing. While not shown in FIG. 24, the tubes were constructed with axial vortex inducers above and below the thermal barrier as described above. The finished tubes were each 4.1 feet in width and 60 feet in length and were filled with water to a 12 inch depth. The growth medium was a modified version of Guillard f/2 medium (Guillard, 1960, J. Protozool. 7:262-68; Guillard, 1975, In Smith and Chanley, Eds. Culture of Marine Invertegrate Animals, Plenum Press, New York; Guillard and Ryther, 1962, Can. J. Microbiol. 8:229-39), containing 22 g/L NaCl, 16 g/L Aquarium Synthetic Sea Salt (Instant Ocean Aquarium Salt, Aquarium Systems Inc., Mentor, Ohio), 420 mg/L NaNO₃, 20 mg/L NaH₂PO₄.H₂O, 4.36 mg/L Na₂EDTA, 3.15 mg/L FeCl₃.6H₂O, 180 μg/L MnCl₂.4H₂O, 22 μg/L ZnSO₄.7H₂O, 10 μg/L CuSO₄.5H₂O, 10 μg/L CoCl₂.6H₂O, 6.3 10 μg/L Na₂MoO₄.2H₂O, 100 μg/L thiamine-HCl, 0.5 μg/L biotin and 0.5 μg/L vitamin B12. A feeder culture of Dunaliella tertiolecta (obtained from the University of Texas, Dr. Jerry Brand) was inoculated into the medium and the algae were allowed to grow and reproduce under ambient light and temperature.

FIG. 18 illustrates an exemplary embodiment of a closed system bioreactor. In this case, the system incorporated two bags, each with a separate roller. The chamber at the upper right of FIG. 18 contained the vortex device, while the chamber at the lower left contained the CO₂ bubbler. Each roller rolled back and forth across a single three layer flexible tube (bag), reversing direction at the end of the tube. Thus, the water periodically reversed flow direction around the closed system.

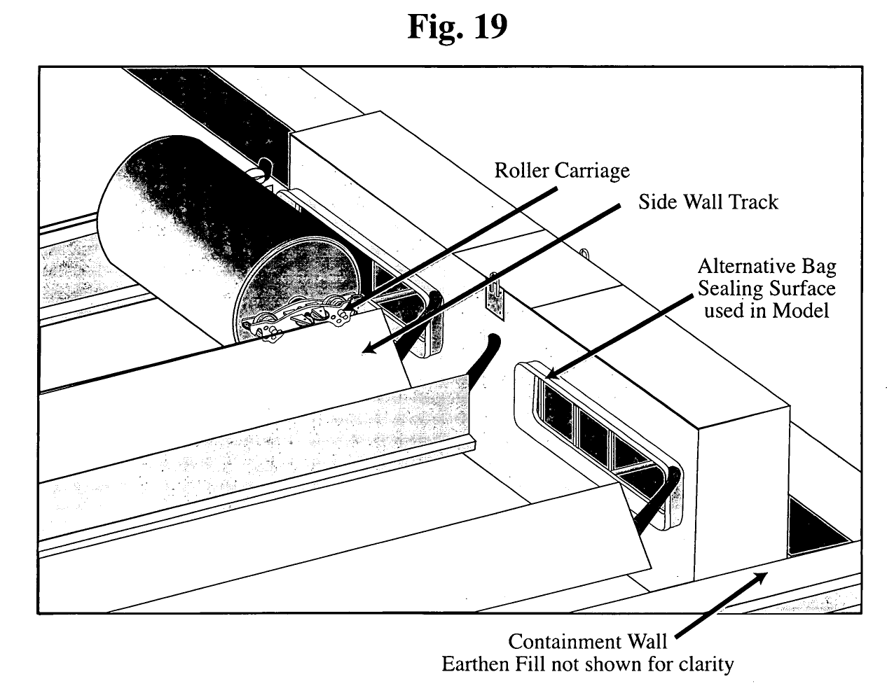

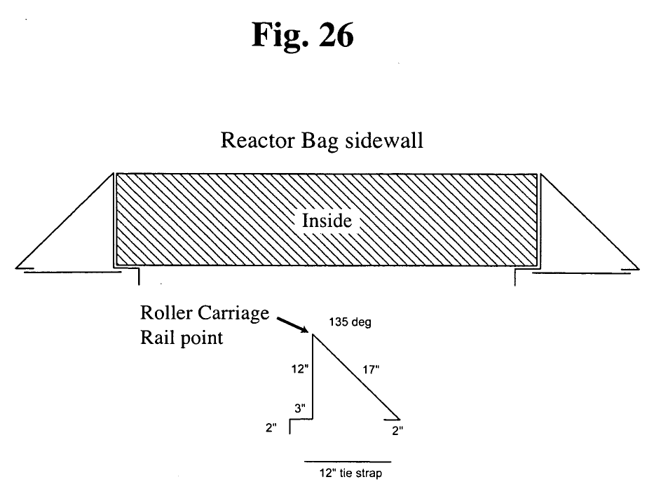

FIG. 19 shows additional details of the roller carriage and support system. The rollers, which were heavy gauge plastic cylinders in this embodiment, were mounted between rolling carriages that rolled on roller sidewall tracks (see FIG. 26), which served to support the carriages and rollers and to maintain them at a constant height above the ground level along the entire length of the tube. The sidewall roller tracks also provided physical support for the sides of the flexible tubes, which might otherwise tend to over strain as they bulged outwards. They further were capable of containing thermal insulation to isolate the flexible tubes from the sides. The supports were made of triangular folded sheet metal 12 inches high with a 3 inch by 2 inch fold that both sits under the edge of the bag and digs into the earth. In another exemplary embodiment for a full scale bioreactor, a concrete sidewall is 36 inches high and 4 inches wide, with 20 inches of the wall buried underground for tipping stability and 2 strands of pre-stressed steel rebar or cable running in the top 25 inches over the entire length to enable dynamic load carrying capacity as the rollers pass.

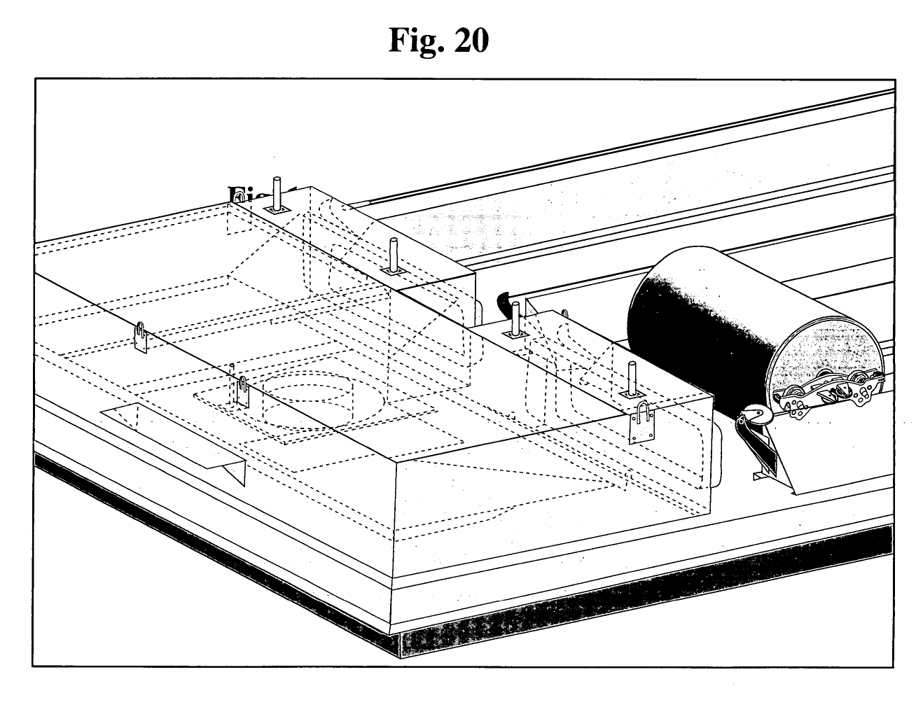

Further details of the exemplary closed bioreactor apparatus are illustrated in FIG. 20, which shows the chamber at the end of the tubes that contains the whirlpool device settled in a square aperture hole in mid-deck. FIG. 20 also shows where the tubes connect to the end chambers through a flange and gasket system, discussed in further detail below. The chamber containing the whirlpool device also contained the actuators for diverting liquid above or below the thermal barrier, discussed in more detail below. The actuated flapper valves comprise the speed-up and slow-down ramps, the ends of which were also attached to actuators to reposition the ramps when the fluid movement direction was reversed. (In the opposite configuration the speed-up ramp becomes a slow down ramp and vice versa.)

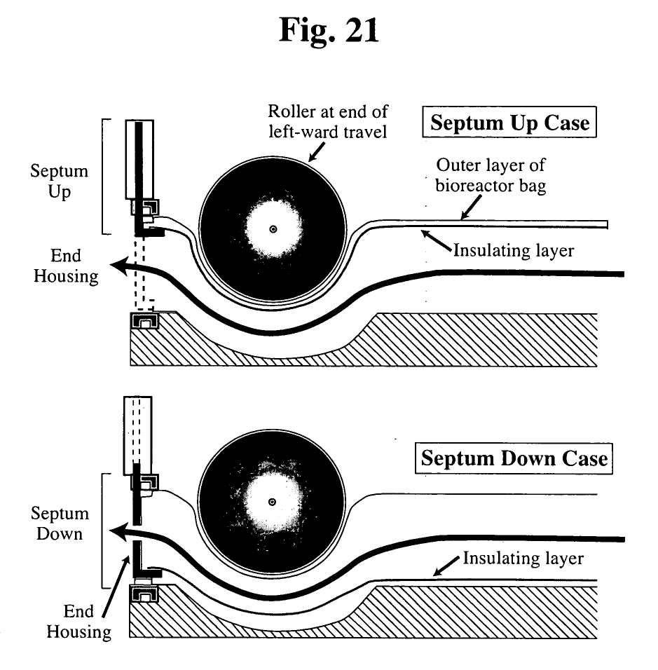

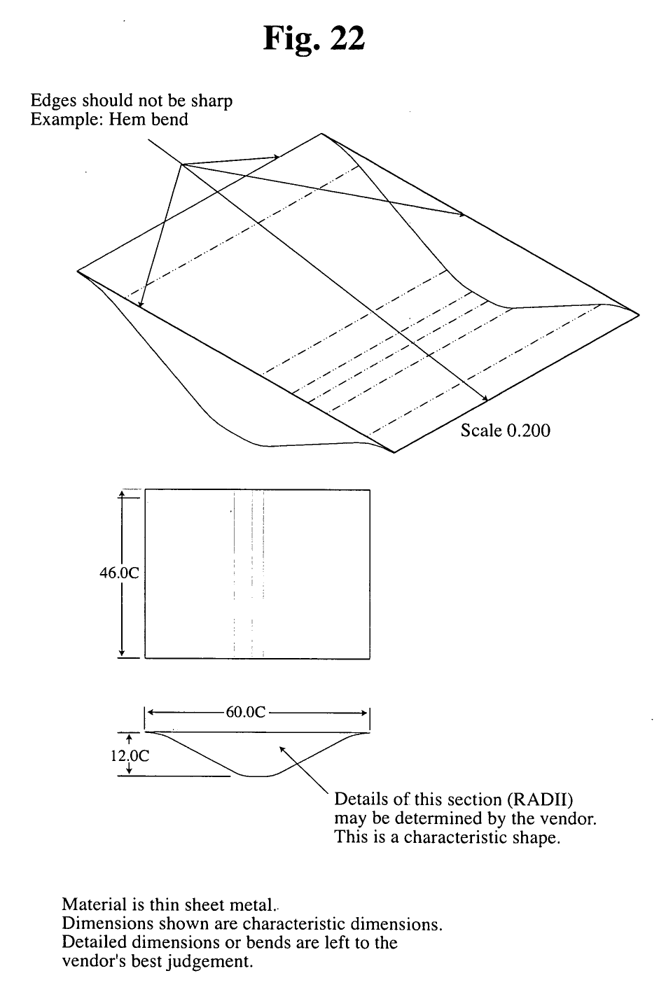

The exemplary closed system bioreactor that was constructed utilized a roller design as illustrated in FIG. 21. This embodiment allowed for reversal of the roller direction and did not require a mechanism for lifting the roller above the housings at the ends of the tubes. The roller was supported at constant height on sidewall roller tracks, as discussed above. Although the ground or other surface was flat and level for almost the entire track length, at the two ends immediately adjacent to the chambers there was a small trenched dip that ran the width of the track. This trench was lined with a metal “belly pan” (FIG. 22) which serves to define the shape of the trench and to prevent soil from entering the bypass area. The trench and belly pan were designed to allow the fluid medium in the tubes to flow under the level of the roller. Because of hydrostatic pressure, the flexible tubes conformed to the ground level and belly pan surface. When the rollers reached the ends of the track, roller movement was stopped by the drive system. The liquid medium was allowed to flow under the rollers into the chambers without resistance from the roller, which was elevated above the liquid flow. This continued flow may be due to inertial momentum or due to the movement of the opposite roller. Due to frictional forces against the thermal barrier, sides of the tubes and components of the chambers, the fluid slowed and ultimately stopped. When fluid flow had reached a sufficiently low velocity, the roller drive was engaged again and the roller moved in the opposite direction. When the first roller stopped over the area of the trench, the second roller engaged the fluid in the tube again and pushed it in the opposite direction, reversing the flow of algae through the system.

FIG. 21 also shows the actuators for diverting water above or below the thermal barrier. As shown, the end of the thermal barrier formed a rigid septum that was attached to a pair of actuators. When the actuators are in the up position, the septum diverted water below the thermal barrier and the barrier floated to the top of the tube. When the actuator was in the down position, fluid was diverted above the thermal barrier, which then sat at the bottom of the tube.

### Example 12

### Whirlpool Device and Inflatable Seal

FIG. 23 shows additional detail of the whirlpool device, located in a chamber or housing at one end of the flexible tubes. Water enters from the right side in this figure, through a bag seal that attaches the tube to the chamber. The thermal barrier septum and attached actuator are also shown on the right, with the septum in a middle position for clarity. In actual operation, the septum would typically be either fully up or down. To the left of the bag seal and septum actuator, water entering the chamber encounters a speed-up ramp, which is attached to a separate actuator. That actuator can alternately position the attached ramp either up or down. When the ramp is down, water entering from the right encounters the ramp. The water is laterally constricted on one side by the side of the chamber and on the other by a central partition that separates the speed-up and slow-down ramps. The water enters at a constant velocity that is determined by the roller tube motion. When it encounters the upward ramp, the height of the water column is decreased from about 12 inches to a lower level, determined by the ramp angle and speed of the water. Because the width of the water column remains the same and the height is diminished, the water flow must increase in velocity as it moves up the ramp, in order to maintain a constant flow of water per unit time. The accelerated water encounters the whirlpool device, which is generally formed as shown in FIGS. 13-15. Water dropping through a central hole in the whirlpool device forms a vortex, resulting in a concentration of lipid-filled algae at the center of the vortex and separation of heavier components of the suspension at the outside of the vortex. However some algae compositions may make the algae heaver than the fluid in which case the algae will be removed from orifices situated around the periphery of the dwell tube as shown in FIG. 15(B). Water traveling down through the central hole encounters a slow-down ramp on the other side of the chamber from the speed-up ramp. The water slows down, enters the second flexible tube and exits the chamber.

FIG. 24 shows an exemplary bag assembly and sealing mechanism. The bag (tube) may be constructed, for example, of top and bottom layers of a thin, high strength, essentially transparent plastic material, such as 0.01 inch thick polyethylene. The thermal barrier may be 0.5 inch or 1.0 inch thick low density poly foam (e.g., foamed polyethylene), in this example with a thin (e.g., 0.0035 inch) facing to decrease algal attachment to the thermal barrier. The thermal barrier may be attached to thinner side strips, which may be attached by thermal adhesive beads or by plastic welding. The sides of the three layers are bonded thermally to create a tube.

The bag (tube) may be stretched over a stiff sealing insert frame inserted into the end of the bag as shown in the drawings of FIG. 24. In full scale systems, the frame may be about 20 feet wide by 12 inches tall and about 6 inches deep axially and may be stiffened by periodic vertical struts along its 20 foot width. A stiffened composite or corrosion resistant metal septum and its alignment and translation mechanism may be incorporated into the frame. The frame and the end of the tube that is stretched over the frame are inserted into an annular pressurized seal that lines the inside of a 12 inch by 20 foot hole in the chamber. Once the frame and bag are inserted into the chamber, the seal is inflated, pressing inwards against and all around the sealing frame and holding the bag and frame securely onto the chamber. The pressurized seal may have redundant expanding pressure seal tubes, each maintained by a separate air compressor and pressure leak alarm sensor. A septum bar may be attached to the septum and then connected to actuators. The installed steptum may be driven up or down by a 4-bar linkage driven by 2 position feedback electro-hydraulic actuators connected by wires to the system controller. Many other actuator systems including common pnuematic linear actuators such as those used in the exemplary model of Example 1 are suitable for moving the septum up and down.

### Example 13

### Biodiesel Production from Algae

Algae are grown to maturity according to Example 11 and harvested for their oil content. A whirlpool device as described in Example 12 is used to partially separate algae from the medium. The algal cell walls are disrupted by passage through a high shear force mechanical device. Oil is separated from other algae contents by centrifugation in a commercial scale centrifuge. The oil is converted into biodiesel by alkaline catalyzed transesterification according to the Connemann process. The amount of biodiesel produced from one bioreactor incorporating two 20 foot×300 foot bioreactor tubes is 2,800 gallons per year.

### Example 14

### Bioreactor Controller

In some embodiments, all aspects of bioreactor function may be controlled by a central processing unit, for example a computer controller. The controller may be operably coupled to various sensors and actuators on the bioreactor. The computer may integrate all functions of bioreactor operation, such as roller movement and alignment, fluid flow, whirlpool operation, harvesting of algae, nutrient and fluid input into the apparatus, gas removal, and CO₂ injection. The computer may operate on a sensing and control program such as LabView made by National Instruments Corporation and may use interface cards and circuits well known in the art to connect with the sensors and actuators of the bioreactor system.

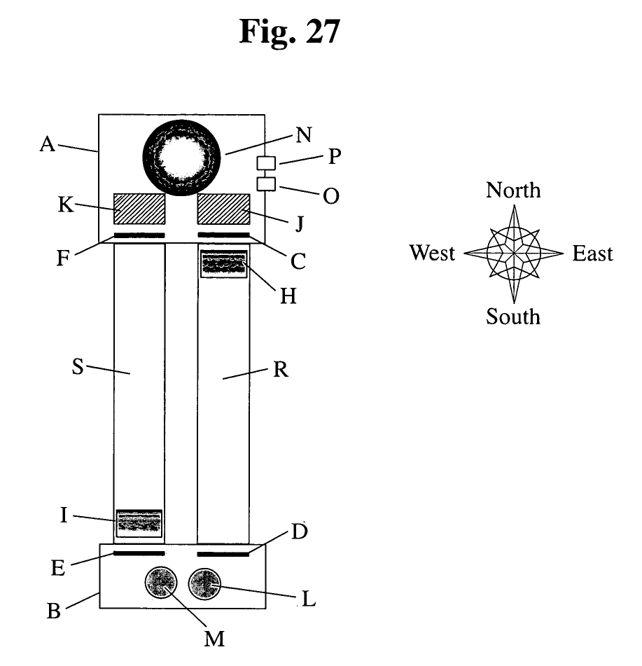

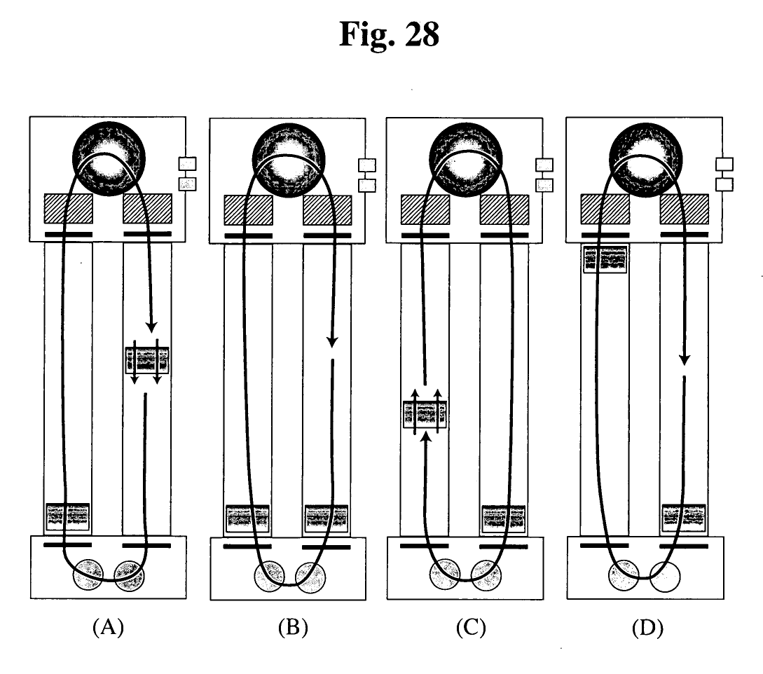

An exemplary operation cycle is illustrated in FIG. 27. The discussion refers to compass directions for clarity, however the skilled artisan will realize that the apparatus in actual use may be aligned in a variety of directions, depending on local geography, solar inclination, temperature, etc. As illustrated in FIG. 27, Rollers H and I are initially positioned over their belly pans at the ends of the tubes. Flapper valve J is in the up position so that water being drawn south comes from the bottom deck of the whirlpool device and flapper valve K is in the down position so that water going north is channeled upward onto the top deck of the whirlpool device. The cycle begins as shown in FIG. 28A with roller H being directed by the controller to begin moving South at a constant speed of 1 foot/second. As it moves, pressure is built up in tube R ahead of roller H and algae growth media (water) begins moving South, westward through the CO2 housing B, then north through tube S, slipping under stationary roller I through the belly pan channel. As the water flows up flapper valve K onto the top deck of A, it begins whirling through the whirlpool N to the bottom deck and expands through flapper valve J to begin backfilling behind roller H.

FIG. 28B shows roller H having fully traversed tube R and having come to a stop at the whirlpool housing. Since both rollers are positioned over belly pans, the liquid is free to continue moving by inertia in the direction shown. With no delay, roller I is caused to begin moving north by the controller as is shown in FIG. 28C. This continues the clockwise flow of the liquid through the whirlpool and back through the CO2 housing as it slips under roller H through the channel created by the belly pan. When roller I finally reaches the whirlpool housing all motion stops except for the fluid media that continues to move clockwise through stored momentum until friction slows the water movement to nearly zero.

At this point the circulation direction of the fluid is reversed. First flapper J is put in the down position so that counterclockwise water flow is directed first onto the top deck and flapper K is in the up position so that exiting lower deck water is expanded into the full height of the bioreactor tube. Roller I starts moving south in under control of the computer, pushing water ahead to start a counter-clockwise fluid movement. After it comes to rest at the end of tube S, roller H immediately starts moving north, to keep the pressure head on the whirlpool and full flow moving. For a short time after roller H comes to rest at the end of tube R, the fluid keeps moving under its own momentum until friction slows it down to near zero speed. Once this is achieved, the controller commands the clockwise motion sequence shown in FIG. 28 to begin again in a constant reciprocating motion. This motion further has the advantages of being inexpensive to implement by not needing to lift the heavy rollers out of the water during turnaround and because of flow reversing is less likely to leave un-turbulent spots in the bioreactor where algae might settle.

The CO₂ injectors may be controlled so that only the bubble injector experiencing counter-current water flow is actuated to take advantage of the increased bubble dwell time and concurrent increased CO₂ absorption (see FIG. 12). The amount of CO₂ injected is not limiting and it is anticipated that CO₂ injection will be intermittent, as determined by medium pH and other indicators.

The septum valves for tube S are E and F. The septum valves for tube R are C and D. Each tube septum may be controlled independently of the other tube septum but each must be coordinated with its roller motion.

Before either roller leaves its rest position the controller must determine whether its associated septum should be placed in the up or down position. If the septum is decided to be in the up position, the septum valve at the roller start position must be in the up position such that water gets drawn under the septum during roller travel. The septum valve at the far end of the tube can be in either position during roller travel as long as the septum valve sealing method allows for expelling water from inside the tube regardless of position. When the roller has stopped however, the septum valve at the far end should be fixed into the upper position.

When the septum is desired to be in the down position, the septum valve at the roller start position must be in the down position so that water is drawn over the top of the septum by roller movement. The septum valve at the far end of the tube can be in either position as long as it is designed to allow the unimpeded expelling of water from either top or bottom tube chamber. When the roller stops however the septum must be fixed into the down position so that water is not allowed to seep under the septum which would allow it to float to the top.

“O” is a fluid temperature sensor interfaced to the computer, which compares the detected temperature with a set point of desired temperature for the algae. Depending on weather and time of day conditions, the computer decides to place the thermal septums in the up or down position and coordinates the actions of the septum valves with the roller movement accordingly. In some cases a sensor may be constructed to determine whether the fluid will gain or lose heat to the temperature and radiative environment. Such a sensor would be constructed by channeling a small amount of fluid (about 0.1 gallon per minute) through a plastic bag of about 3 feet square by 3 inches deep that is laying on ground substantially the same temperature as the ground the main bioreactors are sitting on. Differential temperature sensors with a resolution of 0.02 degree F. measure the temperature at both the intake and outlet of the sensor bag. If the temperature is calculated to be increasing as fluid passes through the bag then the computer positions the septums to expose the fluid to the environment if the fluid is too cold in the bags or to insulate the bags from the environment if the fluid is too warm. The converse logic would apply if the sensor bag indicates that environmental exposure would cool the fluid.

“P” is a pH sensor and is interfaced to the computer. The value of the fluid pH is compared with a desirable pH set point that is indicative of the proper concentration of dissolved CO2 in the water to support optimum growth or harvesting. When the pH is too high the computer opens valves to the appropriate CO2 bubbler to allow pure CO2 or flue gas containing CO2 to bubble through the water making it more acid with the formation of carbonic acid and lowering the pH.

All of the COMPOSITIONS, APPARATUS, SYSTEMS and METHODS disclosed and claimed herein can be made and executed without undue experimentation in light of the present disclosure. While the compositions and methods have been described in terms of preferred embodiments, it is apparent to those of skill in the art that variations maybe applied to the COMPOSITIONS, APPARATUS, SYSTEMS and METHODS and in the steps or in the sequence of steps of the methods described herein without departing from the concept, spirit and scope of the invention. More specifically, certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for algal culture comprising:
a) placing algae in aqueous medium in a closed system bioreactor, the bioreactor comprising one or more flexible tubes operably coupled to one or more peristaltic rollers;
b) exposing the algae to sunlight;
c) culturing the algae under conditions allowing algal reproduction and growth;
d) using the rollers to move the medium through the tubes, wherein the movement of the rollers removes photosynthetic oxygen from the tubes and scrubs the surface of the tubes to reduce biofilm on the tube surfaces.

2. The method of claim 1, wherein the bioreactor comprises 2 tubes, each tube operably coupled to a different roller.

3. The method of claim 1, wherein the tubes comprise a thermal barrier.

4. The method of claim 3, further comprising diverting medium above or below the thermal barrier to regulate the temperature of the medium.

5. The method of claim 1, further comprising separating algae from the medium.

6. The method of claim 5, further comprising removing oil from the algae.

7. The method of claim 6, further comprising producing biodiesel from the oil.

8. The method of claim 7, wherein the biodiesel is produced by transesterification.

9. The method of claim 1, further comprising using axial vortex inducers to induce formation of rotating water columns within the tubes, the rotation of the water columns moving algae between the light-exposed upper region and the darker lower regions of the tube.

10. The method of claim 9, wherein adjacent water columns in the tube rotate in opposite clockwise or counterclockwise directions.

11. The method of claim 1, further comprising introducing CO₂ gas into the medium using one or more CO₂ bubblers.

12. The method of claim 5, wherein algae are partially separated from the medium using a whirlpool device.

13. The method of claim 6, further comprising separating non-oil products from the algae.

14. The method of claim 13, wherein the non-oil products comprise carbohydrates.

15. The method of claim 14, wherein the carbohydrates are converted into hydrogen gas, methane gas and/or ethanol.

16. The method of claim 1, further comprising harvesting the algae for use in animal or human food.

17. The method of claim 16, wherein the algae are Spirulina, Dunaliella or Tetraselmis.

18. The method of claim 1, further comprising using the algae as food for an algae-eating aquatic species.

19. The method of claim 18, wherein the aquatic species is a peneid shrimp.

20. The method of claim 3, wherein the tubes are arranged in a horizontal position on the ground.

21. The method of claim 20, further comprising adjusting the height of the thermal barrier above the ground to control the temperature of the aqueous suspension.

22. The method of claim 21, wherein during daylight hours the flow of the aqueous suspension is directed below the thermal barrier to maintain the temperature of the suspension at ground temperature and above the thermal barrier to warm the suspension.

23. The method of claim 21, wherein during nighttime hours the flow of the aqueous suspension is directed above the thermal barrier to cool the suspension and below the thermal barrier to maintain the temperature of the suspension at ground temperature.

24. The method of claim 2, wherein the rollers reverse direction when they reach the ends of the tubes.

25. The method of claim 24, wherein a dip and belly pan are located below the roller at each end of each tube to allow medium to flow under the roller.

26. The method of claim 2, further comprising controlling the movement of the rollers to prevent skewing.

27. A system for producing biodiesel from algae comprising:
a) a closed bioreactor comprising two flexible tubes operably coupled to two peristaltic rollers, the tubes containing a suspension of algae in aqueous medium;
b) a mechanism for harvesting the algae from the medium;
c) a device for separating oil from the algae;
d) an apparatus for converting the oil into biodiesel.

28. The system of claim 27, wherein the rollers are arranged to roll down the length of the flexible tubes to move the suspension through the tubes.

29. The system of claim 28, wherein the mechanism for harvesting algae comprises a whirlpool device and one or more sipper tubes.

30. The system of claim 29, wherein the whirlpool device comprises a speed-up ramp, a dwell tube and a slowdown ramp.

31. The system of claim 30, wherein the rollers reverse direction at the ends of the tubes.

32. The system of claim 31, wherein the positions of the speed-up and slowdown ramps are reversed when the rollers reverse direction.

33. The system of claim 27, wherein the mechanism for harvesting algae comprises at least one centrifuge.

34. The system of claim 27, wherein the apparatus for converting oil into biodiesel utilizes a transesterification process.

35. The system of claim 28, wherein the rollers in contact with the tubes are arranged so that they compress the tubes to about 85% of the height of the uncompressed tubes.

36. The system of claim 29, wherein movement of the aqueous suspension through the tubes results in the formation of a fluid vortex within the whirlpool device.

37. The system of claim 36, wherein the whirlpool fluid movement results in a partial separation of oil-containing algae from the aqueous medium.

38. The system of claim 27, wherein the tubes contain a thermal barrier arranged horizontally within the tubes, to regulate the temperature of the aqueous suspension within the tubes.

39. The system of claim 27, wherein the outer surface of the tubes is comprised of a plastic.

40. The system of claim 39, wherein the plastic is selected from the group consisting of polyethylene, polypropylene, polyurethane, polycarbonate, polyvinylpyrrolidone, polyvinylchloride, polystyrene, poly(ethylene terephthalate), poly(ethylene naphthalate), poly(1,4-cyclohexane dimethylene terephthalate), polyolefin, polybutylene, polyacrylate and polyvinlyidene chloride.

41. The system of claim 39, wherein the outer surface of the tubes is comprised of 0.01 inch thick polyethylene.

42. The system of claim 38, wherein the thermal barrier comprises a 0.5 to 1.0 inch thick layer of polyethylene foam or other plastic containing an air filled cell construction.

43. The system of claim 38, wherein the upper surface of the thermal barrier comprises a layer of sand, a translucent ceramic or plastic, a silicate or glass.

44. The system of claim 43, wherein the upper surface of the thermal barrier exhibits an infrared emissivity of close to 1.0.

45. The system of claim 28, wherein movement of the rollers along the tubes collects oxygen and other gases from the medium for removal from the system.

46. The system of claim 39, wherein the upper layer of plastic is indented with a linear Frenel pattern that collects sunlight from a lower Snell's law angle and directs it into the algae growing medium.

47. The system of claim 46, where the tubes are laid out perpendicular to the low angle southern sun for the winter months in temperate climates.
